SEQUENCE LISTING (52 sequences)

SEQ ID NO: 1 - protein, 126 aa, Artificial
  Variable chain of single chain antibody.
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYGAFDIWGQGTLVTVSS

SEQ ID NO: 2 - protein, 126 aa, Artificial
  Variable chain of single chain antibody.
EAQLVQSGAEGKKPGSSVKVSCKAPGGTFNSYAISWVRQAPGQGLEWMGGIIPIFGTAYYAQKFQGRVTITADESTSTAYMELSSLRSEDTAVYYCAREEGPYCSSTSCYGAFDIWGRGTLVTVSS

SEQ ID NO: 3 - protein, 126 aa, Artificial
  Variable chain of single chain antibody.
EVQLVQSGAEVKKPGSSVKVSCKASGGTFSSYAIGWVRQAPGQGLEWMGGIIPIFGIANYAQKFQGRVTITADESTSSAYMELSSLRSEDTAVYYCAREEGPYCSSTSCYGAFDIWGQGTLVTVSS

SEQ ID NO: 4 - protein, 126 aa, Artificial; identical to SEQ ID NO: 3
  Variable chain of single chain antibody.

SEQ ID NO: 5 - protein, 126 aa, Artificial
  Variable chain of single chain antibody.
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYAAFDIWGQGTLVTVSS

SEQ ID NO: 6 - protein, 126 aa, Artificial
  Variable chain of single chain antibody.
EVQLVQSGAEVKKPGSSVKVSCKASGGTFSSYAISWVRQAPGQGLEWVGGIIPIFGTANYAQKFQGRVKITADESASTAYMELSSLRSEDTAVYYCAREEGPYCSSTSCYGAFDIWGQGTLVTVSS

SEQ ID NO: 7 - protein, 126 aa, Artificial
  Variable chain of single chain antibody.
EVQLVQSGAEVKKLGSSVKVSCKASGGTFSSYAISWVRQAPGQGLEWVGGIIPIFGTANYAQKFQGRVKITADESASTAYMELSSLRSEDTAVYYCAREEGPYCSSTSCYGAFDIWGQGTLVTVSS

SEQ ID NO: 8 - protein, 126 aa, Artificial; identical to SEQ ID NO: 6
  Variable chain of single chain antibody.

SEQ ID NO: 9 - protein, 126 aa, Artificial; identical to SEQ ID NO: 6
  Variable chain of single chain antibody.

SEQ ID NO: 10 - protein, 126 aa, Artificial; identical to SEQ ID NO: 6
  Variable chain of single chain antibody.

SEQ ID NO: 11 - protein, 126 aa, Artificial; identical to SEQ ID NO: 6
  Variable chain of single chain antibody.

SEQ ID NO: 12 - protein, 108 aa, Artificial
  Variable chain of single chain antibody.
QSVLTQDPAVSVALGQTVKITCQGDSLRSYFASWYQQKPGQAPTLVMYARNDRPAGVPDRFSGSKSGTSASLAISGLQSEDEADYYCAAWDDSLNGYLFGAGTKLTVL

SEQ ID NO: 13 - protein, 108 aa, Artificial; identical to SEQ ID NO: 12
  Variable chain of single chain antibody.

SEQ ID NO: 14 - protein, 108 aa, Artificial
  Variable chain of single chain antibody.
QSVLTQDPAVSVALGQTVKITCQGDSLRSYFASWYQQKPGQAPTLVMYARNDRPAGVPDRFSGSKSGTSASLAISGLQPEDEADYYCAAWDDSLNGYLFGAGTKLTVL

SEQ ID NO: 15 - protein, 108 aa, Artificial; identical to SEQ ID NO: 14
  Variable chain of single chain antibody.

SEQ ID NO: 16 - protein, 108 aa, Artificial
  Variable chain of single chain antibody.
QSVLTQDPAASVALGQTVKITCQGDSLRSYFASWYQQKPGQAPTLVMYARNDRPAGVPDRFSGSKSGTSASLAISGLQSEDEADYYCAAWDDSLNGYLFGAGTKLTVL

SEQ ID NO: 17 - protein, 108 aa, Artificial
  Variable chain of single chain antibody.
QSVLTQDPAVSVALGQTVKITCQGDSLRSYLASWYQQKPGQAPTLVTYARNDRPAGVPDRFSGSKSGTSASLAISGLQSEDEADYYCAAWDDSLNGYLFGAGTKLTVL

SEQ ID NO: 18 - protein, 108 aa, Artificial; identical to SEQ ID NO: 17
  Variable chain of single chain antibody.

SEQ ID NO: 19 - protein, 108 aa, Artificial; identical to SEQ ID NO: 17
  Variable chain of single chain antibody.

SEQ ID NO: 20 - protein, 108 aa, Artificial; identical to SEQ ID NO: 17
  Variable chain of single chain antibody.

SEQ ID NO: 21 - protein, 108 aa, Artificial; identical to SEQ ID NO: 17
  Variable chain of single chain antibody.

SEQ ID NO: 22 - protein, 108 aa, Artificial; identical to SEQ ID NO: 17
  Variable chain of single chain antibody.

SEQ ID NO: 23 - protein, 126 aa, Artificial; identical to SEQ ID NO: 2
  Variable chain of single chain antibody.

SEQ ID NO: 24 - protein, 126 aa, Artificial; identical to SEQ ID NO: 3
  Variable chain of single chain antibody.

SEQ ID NO: 25 - protein, 126 aa, Artificial; identical to SEQ ID NO: 5
  Variable chain of single chain antibody.

SEQ ID NO: 26 - protein, 126 aa, Artificial; identical to SEQ ID NO: 6
  Variable chain of single chain antibody.

SEQ ID NO: 27 - protein, 126 aa, Artificial
  Variable chain of single chain antibody.
EAQLVQSGAEGKKPGSSVKVSCKAPGGTFNSYAISWVRQAPGQGLEWMGGIIPIFGTAYYAQKFQGRVTITADESTSTAYMELSSLRSEDTAVYYCAREEGPYCSSTSCYAAFDIWGRGTLVTVSS

SEQ ID NO: 28 - protein, 126 aa, Artificial
  Variable chain of single chain antibody.
EVQLVQSGAEVKKPGSSVKVSCKASGGTFSSYAIGWVRQAPGQGLEWMGGIIPIFGIANYAQKFQGRVTITADESTSSAYMELSSLRSEDTAVYYCAREEGPYCSSTSCYAAFDIWGQGTLVTVSS

SEQ ID NO: 29 - protein, 126 aa, Artificial
  Variable chain of single chain antibody.
EVQLVQSGAEVKKPGSSVKVSCKASGGTFSSYAISWVRQAPGQGLEWVGGIIPIFGTANYAQKFQGRVKITADESASTAYMELSSLRSEDTAVYYCAREEGPYCSSTSCYAAFDIWGQGTLVTVSS

SEQ ID NO: 30 - protein, 108 aa, Artificial; identical to SEQ ID NO: 12
  Variable chain of single chain antibody.

SEQ ID NO: 31 - protein, 108 aa, Artificial; identical to SEQ ID NO: 14
  Variable chain of single chain antibody.

SEQ ID NO: 32 - protein, 108 aa, Artificial
  Variable chain of single chain antibody.
QSVLTQDPAVSAALGQTVKITCQGDSLRSYFASWYQQKPGQAPTLVMYARNDRPAGVPDRFSGSKSGTSASLAISGLQSEDEADYYCAAWDDSLNGYLFGAGTKLTVL

SEQ ID NO: 33 - protein, 108 aa, Artificial; identical to SEQ ID NO: 17
  Variable chain of single chain antibody.

SEQ ID NO: 34 - protein, 108 aa, Artificial; identical to SEQ ID NO: 12
  Variable chain of single chain antibody.

SEQ ID NO: 35 - protein, 108 aa, Artificial; identical to SEQ ID NO: 14
  Variable chain of single chain antibody.

SEQ ID NO: 36 - protein, 108 aa, Artificial; identical to SEQ ID NO: 17
  Variable chain of single chain antibody.

SEQ ID NO: 37 - protein, 29 aa, Artificial
  EGFR binding motif.
EXXXAXXEIXXLPNLNXXQXXAFIXSLXD

SEQ ID NO: 38 - protein, 29 aa, Artificial
  EGFR binding motif.
EMWXAWXEIRXLPNLNGWQMTAFIXSLLD

SEQ ID NO: 39 - protein, 29 aa, Artificial
  EGFR binding motif.
EXXXAXXEIXXLPNLNGWQMTAFIASLXD

SEQ ID NO: 40 - protein, 29 aa, Artificial
  EGFR binding motif.
EXXXAXXEIGXLPNLNWGQXXAFIXSLWD

SEQ ID NO: 41 - protein, 29 aa, Artificial
  EGFR binding motif.
EXXIAVXEIGELPNLNWGQXDAFINSLWD

SEQ ID NO: 42 - protein, 57 aa, Artificial
  EGFR binding motif.
VDNKFNKEQQNAFYEILHLPNLNEQRNAFIQSLKDDPSQSANLLAEAKKLNDAQAPK

SEQ ID NO: 43 - protein, 5 aa, Artificial
  Peptide translocation signal.
REDLK

SEQ ID NO: 44 - protein, 4 aa, Artificial
  Peptide translocation signal.
REDL

SEQ ID NO: 45 - protein, 4 aa, Artificial
  Peptide translocation signal.
RDEL

SEQ ID NO: 46 - protein, 4 aa, Artificial
  Peptide translocation signal.
KDEL

SEQ ID NO: 47 - DNA, 22 bp, Artificial
  Synthetic oligonucleotide PCR primer.
agtaacgtttgtcagtaattgc

SEQ ID NO: 48 - DNA, 22 bp, Artificial
  Synthetic oligonucleotide PCR primer.
gtcgattttgttacatctacac

SEQ ID NO: 49 - DNA, 21 bp, Artificial
  Synthetic oligonucleotide PCR primer.
ttaagcttctgcaggctagtg

SEQ ID NO: 50 - DNA, 21 bp, Artificial
  Synthetic oligonucleotide PCR primer.
gagaccgaggagagggttagg

SEQ ID NO: 51 - DNA, 21 bp, Artificial
  Synthetic oligonucleotide PCR primer.
agccgcatagcagctggtact

SEQ ID NO: 52 - DNA, 30 bp, Artificial
  Synthetic oligonucleotide PCR primer.
accagctgctatgcggcttttgatatctgg

(19) United States
(12) Patent Application Publication
(10) Pub. No.: US 2010/0009390 A1
(43) Pub. Date: Jan. 14, 2010

(54) MUTANT ANTIBODIES WITH HIGH AFFINITY FOR EGFR

(75) Inventors: JAMES D. MARKS, Kensington, CA (US); YU ZHOU, San Francisco, CA (US)

Correspondence Address: Weaver Austin Villeneuve & Sampson LLP, P.O. BOX 70250, OAKLAND, CA, 94612-0250, US

(73) Assignee: THE REGENTS OF THE UNIVERSITY OF CALIFORNIA, Oakland, CA (US)

(21) Appl. No.: 12/463,123

(22) Filed: May 8, 2009

Related U.S. Application Data
(63) Continuation of application No. 61/052,050, filed on May 9, 2008.

(51) Int. Cl. G01N 33/574; C07K 16/30; C12N 5/00

(52) U.S. Cl. 435/7.23; 530/387.3; 435/377

(57) ABSTRACT

This invention provides antibodies that have improved affinity for the epidermal growth factor receptor (EGFR). In addition, this invention provides microparticles and nanoparticles comprising a plurality of EGFR affinity moieties that are effectively internalized by cells expressing an EGFR.

### CROSS-REFERENCE TO RELATED APPLICATIONS

This application claims benefit of and priority to U.S. Ser. No. 61/052,050, filed on May 9, 2008 which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported in part by Grant No: P50 CA58207 from the National Institutes of Health. The government of the United States of America has certain rights in this invention.

### FIELD OF THE INVENTION

This invention pertains to the filed of cancer therapeutics and diagnostics. In particular novel antibodies are provided that bind the epidermal growth factor receptor (EGFR) as well as nanoparticles and microparticles that are effectively internalized by cells expressing or over-expressing EGFR.

### BACKGROUND OF THE INVENTION

The epidermal growth factor receptor (EGFR) is a transmembrane receptor involved in cell proliferation, growth, migration, invasion, and survival. The receptor is structurally composed of three principal domains: an extracellular ligand-binding domain, a transmembrane domain, and an intracellular domain with intrinsic tyrosine kinase (TK) activity. Binding of activating ligands to the extracellular domain induces receptor homo- or hetero-dimerization, resulting in activation of the TK domain moiety, autophosphorylation, and activation of the downstream signaling pathways.

A wide range of tumors over-express epidermal growth factor receptor (EGFR), including breast, lung, colorectal, and brain cancers (Laskin and Sandler (2004) Cancer Treat. Rev. 30:1-17; Nicholson et al. (2001). Eur. J. Cancer, 37: S9-S15). EGFR (vIII), a truncated form of EGFR, is found in glioblastomas (Kuan et al. (2001) Endocr. Relat. Cancer, 8: 83-96; Friedman and Bigner (2005) Engl. J. Med. 353: 1997-1999), but not in normal tissues, making it plausible to target tumors expressing this variant with a greater degree of specificity. Certain monoclonal antibodies targeting the extracellular domain (ECD) of EGFR and small-molecule inhibitors of tyrosine kinase activity have been evaluated in clinical trials and approved for clinical use (Grunwald and Hidalgo (2003) J. Natl Cancer Inst. 95: 851-867; Mendelsohn and Baselga (2003) J. Clin. Oncol. 21: 2787-2799). While these antibodies have demonstrated clinically important response rates, the percentage of patients with metastatic disease who responded, and the duration of their responses, is modest (Baselga et al. (1996) J. Clin. Oncol. 14: 737-744; Cobleigh et al. (1999) J. Clin. Oncol. 17: 2639-2648; Vogel et al. (2002) J. Clin. Oncol. 20: 719-726).

An alternative approach that could show greater efficacy consists of using antibodies to target chemotherapeutic agents or toxins specifically to tumor cells overexpressing EGFR or EGFR (vIII). Internalization, not simply binding, is a known requisite for optimal activity of many such drug delivery strategies (Noble et al. (2004) Expert Opin. Ther. Targets, 8: 335-353). Methods for the generation of internalizing antibodies have expanded with the availability of display technologies (Becerril et al. (1999) Biochem. Biophys. Res. Commun. 255: 386-393; Nielsen and Marks (2000) Pharm. Sci. Technol. Today, 3: 282-291; Poul et al. (2000) J. Mol. Biol. 301: 1149-1161; Heitner et al. (2001) J. Immunol. Methods, 248: 17-30; Wang and Shusta (2005) J. Immunol. Methods, 304: 30-42). For example, internalizing human antibodies against ErbB2 and EGFR have been generated by direct selection of non-immune phage antibody libraries (Sheets et al. (1998) [published erratum appears in Proc Natl Acad Sci USA 1999 January 1996(2):795]. Proc. Natl. Acad. Sci. USA, 95: 6157-6162; O'Connell (2002) J. Mol. Biol. 321: 49-56) on live cells overexpressing ErbB2 or EGFR (Poul et al. (2000) J. Mol. Biol. 301: 1149-1161; Heitner et al. (2001) J. Immunol. Methods, 248:17-30).

Liposomal and immunoliposomal drug delivery have resulted in an improved therapeutic index for a variety of small-molecule therapeutic drugs (Noble et al. (2004) Expert Opin. Ther. Targets, 8: 335-353; Drummond et al. (1999) Pharmacol. Rev. 51: 691-743; Drummond et al. (2006) Cancer Res. 66: 3271-3277; Sapra and Allen (2003) Prog. Lipid Res. 42: 439-462). An anti-EGFR immunoliposome constructed with a high-affinity anti-EGFR antigen binding fragment (Fab)-targeting ligand derived from Cetuximab (C225 IgG, Imclone) demonstrated efficient drug delivery and activity in cell culture (Mamot et al. (2003) Cancer Res. 63: 3154-3161), and in in vivo tumor xenograft models (Mamot et al. (2005) Cancer Res. 65: 11631-11638).

While C225 binds EGFR with high affinity (KD=0.5 nM), human antibody fragments isolated from non-immune phage libraries typically have considerably lower affinities (Sheets et al. (1998) [published erratum appears in Proc Natl Acad Sci USA 1999 January 1996(2):795]. Proc. Natl. Acad. Sci. USA, 95: 6157-6162; Marks et al. (1991) J. Mol. Biol. 222: 581-597). It is possible to increase antibody affinity significantly using molecular evolution and display technologies (Marks et al. (1992) Biotechnology (NY), 10: 779-783; Schier et al. (1996) J. Mol. Biol. 255: 28-43); however, it has not been determined whether intrinsic antibody affinity has any substantial effect on cellular uptake of any nanoparticles, including immunoliposomes.

### SUMMARY

In certain embodiments this invention provides mutant antibodies (C10 mutants) that have improved affinity for the epidermal growth factor receptor (EGFR). In certain embodiments the antibody has an KD for EGFR of less than 260 nM, preferably less than about 200 nM, 150 nM, 100 nM, or 50 nM, more preferably less than about 40 nM, 30 nM, 20 nM, 15 nM, 10 nM, 5 nM or 1 nM. In certain embodiments the antibody comprises a heavy chain variable domain (VH) comprising one, two, or all three VH CDRs of an antibody selected from the group consisting of P2/1, P2/2, P2/3, P2/4, P2/5, 2124, 2224, 3524, P3/1, P3/2, P3/3, P3/4, and P3/5, in certain embodiments, more preferably from the group consisting of P2/1, P2/2, P2/3, P2/4, 2124, 2224, and 3524. In certain embodiments the antibody comprises a light chain variable domain (VL) comprising one, two, or all three VL CDRs of an antibody selected from the group consisting of P2/1, P2/2, P2/3, P2/4, P2/5, 2124, 2224, 3524, P3/1, P3/2, P3/3, P3/4, and P3/5, in certain embodiments more preferably of an antibody selected from the group consisting of P2/5, and P3/5. In certain embodiments the antibody comprises the three VH CDRs and the three VL CDRs of an antibody selected from the group consisting of P2/1, P2/2, P2/3, P2/4, P2/5, 2124, 2224, 3524, P3/1, P3/2, P3/3, P3/4, and P3/5, in certain embodiments more preferably of an antibody selected from the group consisting of P2/1, P2/2, P2/3, P2/4, P2/5, 2124, 2224, 3524, P3/1, and P3/5. In certain embodiments the antibody comprises the VH domain and the VL domain of an antibody selected from the group consisting of P2/1, P2/2, P2/3, P2/4, P2/5, 2124, 2224, 3524, P3/1, P3/2, P3/3, P3/4, and P3/5, in certain embodiments more preferably of an antibody selected from the group consisting of P2/1, P2/2, P2/3, P2/4, P2/5, 2124, 2224, 3524, P3/1, and P3/5. In various embodiments the antibody is an antibody selected from the group consisting of an scFv, an IgG, a Fab, an (Fab′)₂, and an (scFv′)₂.

In various embodiments the antibody is coupled to an effector thereby forming a chimeric moiety In certain embodiments the effector comprises a moiety selected from the group consisting of a nanoparticle or microparticle, a microcapsule, a cytotoxin, a detectable label, a radionuclide, a drug, a liposome, a ligand, and an antibody. In certain embodiments the effector comprises a moiety selected from the group consisting of a liposome, and a polymeric nanoparticle. In certain embodiments the effector comprises a liposome containing a moiety selected from the group consisting of an anti-cancer drug, a detectable label, and a radiosensitizing agent. In certain embodiments the effector comprises a lipidic microparticle or nanoparticle (e.g., a liposome, a lipid-nucleic acid complex, a lipid-drug complex, a solid lipid particle, a microemulsion droplet, etc.). In certain embodiments the microparticle or nanoparticle is a micelle. In certain embodiments the microparticle or nanoparticle comprises a pharmaceutical. In certain embodiments the microparticle or nanoparticle is a liposome (e.g., a multilamellar liposome or a unilamellar liposome). The liposome can, optionally, be a stearically stabilized liposome. In certain embodiments the liposome or other lipidic particle contains an anti-cancer pharmaceutical or an anti-cancer siRNA. In certain embodiments the effector comprises a polymeric nanoparticle. In various embodiments the nanoparticle comprises an anti-cancer pharmaceutical or an anti-cancer siRNA.

In certain embodiments methods are provided for inhibiting the growth and/or proliferation of a cancer cell expressing an epidermal growth factor receptor (EGFR). The methods typically involve contacting the cell with a composition comprising an antibody that binds an EGFR receptor and/or a chimeric moiety comprising an antibody that binds an EGFR receptor attached to an effector as described herein. In certain embodiments the contacting comprises systemic administration to a mammal (e.g., a human or a non-human mammal) in need thereof. In certain embodiments the contacting comprises administration to the tumor or tumor site. In certain embodiments the contacting comprises postoperative administration to a tumor site. In certain embodiments the cancer is selected from the group consisting of glioblastoma, breast cancer, bladder cancer, cervical cancer, kidney cancer, ovarian cancer, squamous cell carcinoma, laryngeal cancer, pancreatic cancer, prostate cancer, and non-small-cell lung cancer. In various embodiments the antibody and/or chimeric moiety is provided in a pharmaceutically acceptable excipient (e.g., as a unit dosage formulation).

In certain embodiments, methods are provided for detecting a cell expressing an epidermal growth factor receptor (EGFR) in a mammal. The methods typically involve contacting the cell with a composition comprising an antibody that binds an EGFR receptor, where the antibody comprises an antibody, e.g., as described above, or a chimeric moiety as described above, where the antibody is attached to an epitope tag or a detectable label; and detecting the epitope tag or the detectable label. In certain embodiments, the detecting, comprises contacting the epitope tag with a labeled moiety that binds to the epitope tag, and detecting the bound label. In certain embodiments, the detecting, comprises detecting the detectable label. In certain embodiments, the contacting comprises systemic administration of the antibody to the mammal.

Also provided in certain embodiments, are methods of delivering an effector into a cell expressing an epidermal growth factor receptor (EGFR). The methods typically involve contacting the cell with a composition comprising an effector attached to an antibody as described herein where the antibody is attached to the effector and whereby the antibody (and the effector) are internalized into the cell. In various embodiments, the effector comprises a moiety selected from the group consisting of a polymeric nanoparticle, a microcapsule, a cytotoxin, a radionuclide, a drug, a liposome, a ligand, and an antibody. In certain embodiments, the effector comprises a moiety selected from the group consisting of a liposome, and a polymeric nanoparticle. In certain embodiments, the effector comprises an immunoliposome containing a moiety selected from the group consisting of an anti-cancer drug, a detectable label, and a radiosensitizing agent. In certain embodiments, the effector comprises an immunoliposome containing an anti-cancer drug or an anti-cancer siRNA.

In still other embodiments, a composition is provided comprising a microparticle or nanoparticle having attached thereto a plurality of affinity moieties that bind to the EGF receptor on a living cell, where the affinity moieties bind to the EGF receptor with a Kd of less than about 270 nM, and the microparticle or nanoparticle has an average of at least 30 binding moieties per particle (or a density of at least 30 moieties per surface area of 100 nm liposome), and where when the nanoparticle is contacted with the cell under conditions that permit endocytosis, the nanoparticle is internalized into the cell. In certain embodiments the average number of affinity moieties (e.g., antibodies) per microparticle or nanoparticle ranges from about 30 to about 200 per particle (or a density ranging from about 30 to about 200 moieties per surface area of 100 nm liposome)) and the affinity of the antibodies for an EGFR on a cell ranges from about 500 nM to about 0.5 nM. In certain embodiments the Kd of the affinity moieties is less than about 263 nM or 264 nM and the microparticles or nanoparticles bear an average of at least about 74 affinity moieties per particle (or a density of at least about 74 moieties per surface area of 100 nm liposome). In certain embodiments the microparticles or nanoparticles bear an average of at least about 148 affinity moieties per particle (or a density of at least about 148 moieties per surface area of 100 nm liposome). In certain embodiments the Kd of the affinity moieties is less than about 15 nM and the microparticles or nanoparticles bear an average of at least about 37 affinity moieties per particle (or a density of at least about 37 moieties per surface area of 100 nm liposome). In certain embodiments the microparticles or nanoparticles bear an average of at least about 74 affinity moieties per particle (or a density of at least about 74 moieties per surface area of 100 nm liposome). In certain embodiments the Kd of the affinity moieties is less than about 0.94 nM and the microparticles or nanoparticles bear an average of at least about 25 affinity moieties per particle (or a density of at least about 25 moieties per surface area of 100 nm liposome). In certain embodiments the microparticles or nanoparticles bear an average of at least about 37 affinity moieties per particle (or a density of at least about 37 moieties per surface area of 100 nm liposome). In certain embodiments the affinity of the affinity moieties ranges from about 270 nM to about 0.5 nM. In certain embodiments the affinity (KD) of the affinity moieties for EGFR ranges from about 50 nM to about 0.8 nM. In certain embodiments the affinity moiety is monovalent. In certain embodiments the affinity moiety is an antibody. In various embodiments the microparticle is a lipidic microparticle. In certain embodiments, the microparticle or nanoparticle is selected from the group consisting of a liposome, a lipid-nucleic acid complex, a lipid-drug complex, a solid lipid particle, and a microemulsion droplet. In certain embodiments, the microparticle or nanoparticle is a micelle. In certain embodiments, the microparticle or nanoparticle comprises a pharmaceutical or a nucleic acid. In certain embodiments, the microparticle or nanoparticle is a liposome (e.g., a multilamellar liposome, a unilamellar liposome). In certain embodiments the liposome is stearically stabilized. In certain embodiments, the liposome contains an anti-cancer pharmaceutical or an anti-cancer siRNA. In various embodiments, the microparticle or nanoparticle is a polymeric nanoparticle that can optionally comprise an anti-cancer pharmaceutical, an anti-cancer siRNA, or another active agent. In certain embodiments, the affinity moiety is an antibody, more preferably a C10 antibody or a mutant C10 antibody, e.g., as described herein.

In still other embodiments, compositions are provided comprising a microparticle or nanoparticle bearing on the surface thereof a plurality of affinity moieties having affinity to EGF receptor on the surface of a living cell, the affinity characterized by Kd of the affinity moieties of less than about 264 nM, where the affinity moiety binds to the epitope also bound by the C10 antibody, and where when the nanoparticle is contacted with the cell under conditions permitting endocytosis, the microparticle or nanoparticle is internalized into the cell. In certain embodiments, the affinity moiety comprises a polypeptide having the amino acid sequence of an antibody selected from the group consisting of P2/1, P2/2, P2/3, P2/4, P2/5, 2124, 2224, 3524, P3/1, P3/2, P3/3, P3/4, and P3/5, having conservative substitutions, or sequences having at least 70% homology with any of the CDRs of the antibodies as determined by a BLAST algorithm. In various embodiments, the microparticle or nanoparticle is a lipidic microparticle or nanoparticle or a polymeric nanoparticle or microparticle as described herein. In various embodiments, the microparticle comprises an anti-cancer pharmaceutical, an anti-cancer siRNA, or other active agent, e.g., as described herein.

In various embodiments, methods are provided for administering (delivering) a pharmaceutical (or nucleic acid or other effector). The methods typically involve administering to a subject in need thereof an effective amount of a microparticle and/or nanoparticle composition as described herein where the microparticle or nanoparticle comprising comprises a pharmaceutical (or nucleic acid or other effector). In certain embodiments, the subject is a human or a non-human mammal diagnosed with cancer. In certain embodiments, the administering comprises a modality selected from the group consisting of systemic administration, inhalation, injection, and administration to an operative site.

### DEFINITIONS

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide. Preferred “peptides”, “polypeptides”, and “proteins” are chains of amino acids whose a carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to peptide mimetics such as amino acids joined by an ether as opposed to an amide bond.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes whole antibodies, antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8: 1323-1331), and in addition to monospecific antibodies, also include bispecific, trispecific, quadraspecific, and generally polyspecific antibodies (e.g., bs scFv).

With respect to antibodies of the invention, the term “immunologically specific” “specifically binds” refers to antibodies that bind to one or more epitopes of a protein of interest (e.g., EGFR ECD), but which do not substantially recognize and bind other molecules in a sample containing a mixed population of antigenic biological molecules. In certain embodiments refer to moieties that bind to the target (e.g., EGFR) with a dissociation constant (Kd) of less than about 1000 nM, preferably less than about 265 nM, more preferably less than about 100 nM, still more preferably less than about 50 nM, even more preferably less than about 25, 20, 15, 10, 5, or 1 nM.

An EGFR affinity moiety is a moiety that specifically binds (within the meaning earlier explained in the context of an antibody) to epidermal growth factor receptor (EGFR), preferably to the EGFR extracellular domain and/or receptor binding site. An EGFR affinity moiety typically binds to EGFR with a Kd of less than about 1000 nM, preferably less than about 265 nM, more preferably less than about 100 nM, still more preferably less than about 50 nM, even more preferably less than about 25, 20, 15, 10, 5, or 1 nM. An EGFR affinity moiety may be a natural or synthetic ligand for EGF receptor, an enzyme, a hormone, a lectin, or any natural, synthetic or recombinant polypeptide, polynucleotide, polysaccharide or a small molecule compound know to bind to EGFR, or artificially selected for binding to EGFR by any known methods. One advantageous method for selection of EGFR affinity moieties is selection of display libraries, for example, of phage display libraries, as explained below. Another known technique for selecting binding polynucleotides (aptamers) is SELEX®. In one preferred embodiment, the EGFR affinity moiety is an antibody, in particular, a single chain Fv antibody fragment

The term “bispecific antibody” as used herein refers to an antibody comprising two antigen-binding sites, a first binding site having affinity for a first antigen or epitope and a second binding site having binding affinity for a second antigen or epitope distinct from the first.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. With respect to the peptides of this invention sequence identity is determined over the full length of the peptide.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendrogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The phrase “specifically target/deliver” when used, for example with reference to a chimeric moiety of this invention refers to specific binding of the moiety to a target (e.g., a cell overexpressing the target protein(s)) this results in an increase in local duration and/or concentration of the moiety at or within the cell as compared to that which would be obtained without “specific” targeting. The specificity need not be absolute, but simply detectably greater/measurably avidity/affinity than that observed for a cell expressing the target protein(s) at normal (e.g., wildtype) or than that observed for a cell that does not express the target protein(s).

Amino acid residues are identified in the present application according to standard 3-letter or 1-letter abbreviations (e.g., as set forth in WIPO standard ST 25.

An “isolated antibody” refers to an antibody that at some time has existed outside an animal typically a mammal. Thus “isolated” excludes naturally occurring antibodies that have existed only in vivo. Alternatively, this term may refer to an antibody that has been sufficiently separated from other proteins or other biomolecules with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example pharmaceutically acceptable preparations.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “anti-cancer drug” is used herein to refer to one or a combination of drugs conventionally used to treat cancer. Such drugs are well known to those of skill in the art and include, but are not limited to doxirubicin, vinblastine, vincristine, taxol, etc.

The term “nanoparticle” refers to a particle having a sub-micron (μm) size. In various embodiments, microparticles have a characteristic size (e.g., diameter) less than about 1 μm, 800 nm, or 500 nm, preferably less than about 400 nm, 300 nm, or 200 nm, more preferably about 100 nm or less, about 50 nm or less or about 30 or 20 nm or less.

The term “microparticle” refers to a particle having a characteristic size of between about 1 μM and 100 μM.

In certain embodiments, the density of affinity moieties (e.g., C10 mutant antibodies) is referred to as a number per particle (e.g., nanoparticle or microparticle). In such cases, the number when viewed as a density is that number of affinity moieties per surface area of a unilamellar phosphatidylcholine-cholesterol liposome having about 80,000 phospholipid molecules and an average surface area per phospholipid molecule of about 0.6 nm² (see e.g., Provoda et al. J. Biol. Chem. 2003, v. 278, p. 35102-35108), giving, in view of the bilayer character of the liposome membrane, the surface area of 24,000 nm². A person skilled in the art will routinely transform the surface density so presented into any other desirable units or expressions for surface density of affinity moieties borne by the particle

In certain embodiments, conservative substitutions of the amino acids comprising any of the antibody sequences, especially CDR regions of such sequence sequences described herein are contemplated. In various embodiments one, two, three, four, or five different residues are substituted. The term “conservative substitution” is used to reflect amino acid substitutions that do not substantially diminish the activity (e.g., EGFR affinity) of the molecule. Typically conservative amino acid substitutions involve substitution one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K), Histidine (H); 5) Isoleucine (I), Leucine (L), Methionine (M), Valine (V); and 6) Phenylalanine (F), Tyrosine (Y), Tryptophan (W).

### BRIEF DESCRIPTION OF THE DRAWINGS

FIG. 1 illustrates the selection of higher affinity scFv by fluorescent activated cell sorting. Yeast displaying scFv were stained with biotinylated EGFR-ECD at the indicated concentrations. The sort gate was set to capture yeast cells with higher binding affinity (gate P2) and better scFv expression (gate P3). The number inside the P2 and P3 gates indicates the percentage of cells within that gate.

FIG. 2 illustrates the binding of yeast-displayed scFv to 1 μM biotinylated EGFR-ECD. (Panel a) Parental C10 clone. (Panel b) Polyclonal yeast (P2 poly) and monoclonal yeast (P2/1, P2/2, P2/3, P2/4, P2/5) from the P2 gate sorting. (Panel c) Polyclonal yeast (P3 poly) and monoclonal yeast (P3/1, P3/2, P3/3, P3/4, P3/5) from the P3 gate sorting.

FIG. 3 shows the deduced amino acid sequences of C10 scFv and the affinity-matured mutants. C10 (SEQ ID NO:1), P2/1 (SEQ ID NO:2), P2/2 (SEQ ID NO:3), P2/3 (SEQ ID NO:4), P2/4 (SEQ ID NO:5), P2/5 (SEQ ID NO:6), P3/1 (SEQ ID NO:7), P3/2 (SEQ ID NO:8), P3/3 (SEQ ID NO:9), P3/4 (SEQ ID NO:10), P3/5 (SEQ ID NO:11).

FIG. 4 shows differential binding of scFv to EGFR positive and negative cell lines as determined by flow cytometry. C10 scFv and C10 mutant scFv stained both EGFR and EGFR vIII positive cells (A431, MDAMB468, and NR6M) but did not stain EGFR negative cells (MDAMB453 and NR6).

FIGS. 5A and 5B illustrate the effect of intrinsic antibody affinity on internalization of EGFR-targeted ILs. (FIG. 5A) Internalization of EGFR-targeted ILs with different affinity compared to non-targeted liposomes in EGFR-over-expressing cell MDAMB468 as determined by fluorescent microscopy. (FIG. 5B) Uptake of EGFR ILs into MDAMB468 cells as determined by flow cytometry.

FIGS. 6A, 6B, and 6C show the effect of scFv affinity and scFv surface density on internalization of EGFR-targeted ILs. (FIG. 6A) Uptake of EGFR ILs with different scFv surface densities into MDAMB468 cells as determined by flow cytometry. Each datum point represents the mean of three independent measurements. (FIG. 6B) Apparent KD of ILs with a surface density of 74 scFv/liposome for MDAMB468 cells. (FIG. 6C) Uptake of EGFR ILs with different scFv surface densities into MDAMB231 cells compared to A431 cells as determined by flow cytometry.

FIG. 7 shows the effect of EGF on the binding and uptake of EGFR scFv antibodies and ILs, C10 (♦), P2/4 (▪), and 2224 ((▴). Effect of increasing EGF concentration (Panel a) on the binding of EGFR scFv to MDAMB468 cells, (Panel b) on the binding of EGFR ILs to MDAMB468 cells, (Panel c) on the uptake of EGFR ILs into MDAMB468 cells, and (Panel d) on the binding of EGFR ILs to U87vIII cells.

FIGS. 8A and 8B illustrate the effect of intrinsic antibody affinity on EGFR ILs cytotoxicity. Cytotoxicity of anti-EGFR immunoliposomal topotecan in (FIG. 8A) EGFR-over-expressing MDAMB468 breast carcinoma and (FIG. 8B) U87vIII glioblastoma. Immunoliposomes constructed with the P2/4 (♦) and 2224 (▪) mutants were compared to those prepared using the parental C10 scFv (▴), non-targeted liposomal topotecan (x), and free topotecan controls (). Data are mean±SD (bars).

FIGS. 9A and 9B illustrate the sequences of VH domains (FIG. 9A) of P2/1 (SEQ ID NO:12), P2/2 (SEQ ID NO:13), P2/4 (SEQ ID NO:14), P3/5 (SEQ ID NO:15), 2124 (SEQ ID NO:16), 2224 (SEQ ID NO:17), and 3524 (SEQ ID NO:18), and VL domains (FIG. 9B) of P2/1 (SEQ ID NO:19), P2/2 (SEQ ID NO:20), P2/4 (SEQ ID NO:21), P3/5 (SEQ ID NO:22), 2124 (SEQ ID NO:23), 2224 (SEQ ID NO:24), and 3524 (SEQ ID NO:25) antibodies.

### DETAILED DESCRIPTION

In certain embodiments this invention pertains to the discovery of novel mutants of the C10 antibody that bind the epidermal growth factor receptor (EGFR) with high affinity. Since the EGFR is upregulated on a number of cancer cells including, but not limited to glioblastoma (e.g., glioblastoma multiforme), breast cancer, bladder cancer, cervical cancer, kidney cancer, ovarian cancer, squamous cell carcinoma, laryngeal cancer, and non-small-cell lung cancer, the antibodies can be used alone to inhibit growth and/or proliferation of these cells, or can be attached to a microparticle or nanoparticle moiety containing an active agent to deliver that active agent to the cells. In certain embodiments the antibodies can be attached directly or though a linker to the active agent and thereby omit the particular component.

It was also discovered that in certain embodiments, the attachment of multiple EGFR affinity moieties (e.g., C10 mutant antibodies, affibodies, etc.) to an effector (e.g., a nanoparticle) can significantly enhance internalization into a cell expressing the EGFR. Moreover, where certain densities of affinity moieties per nanoparticle are maintained it is possible to achieve a high level of internalization even with lower affinity moieties.

Thus, in certain embodiments, this microparticles and/or nanoparticles are provided having attached thereto EGFR affinity moieties in particular numbers/densities for certain affinity moieties, as explained herein.

The antibodies and the affinity moiety/particle chimeric moieties thereby provide a effective means of delivering an active agent to a cell expression EGFR.

### I. Affinity Moieties.

In certain embodiments, compositions are provided typically comprising a plurality of affinity moieties that bind the epidermal growth factor receptor (EGFR) attached to a microparticle or nanoparticle, e.g., as described above.

A number of affinity moieties that bind to the extracellular domain of the EGFR are known to those of skill in the art. Such affinity moieties include, but are not limited to anti-EGFR antibodies, EGFR binding peptides, anti-EGFR affibodies, EGFR receptor ligands, and the like.

A) EGFR Binding Peptides.

A number of EGFR binding peptides are known to those of skill in the art. Thus for example PCT Publication (PCT/EP2006/011669, WO 2007/065635 A1, which is incorporated herein by reference in its entirety) discloses an epidermal growth factor receptor (EGFR) binding polypeptide, comprising an epidermal growth factor receptor binding motif of the formula:

(SEQ ID NO :26)

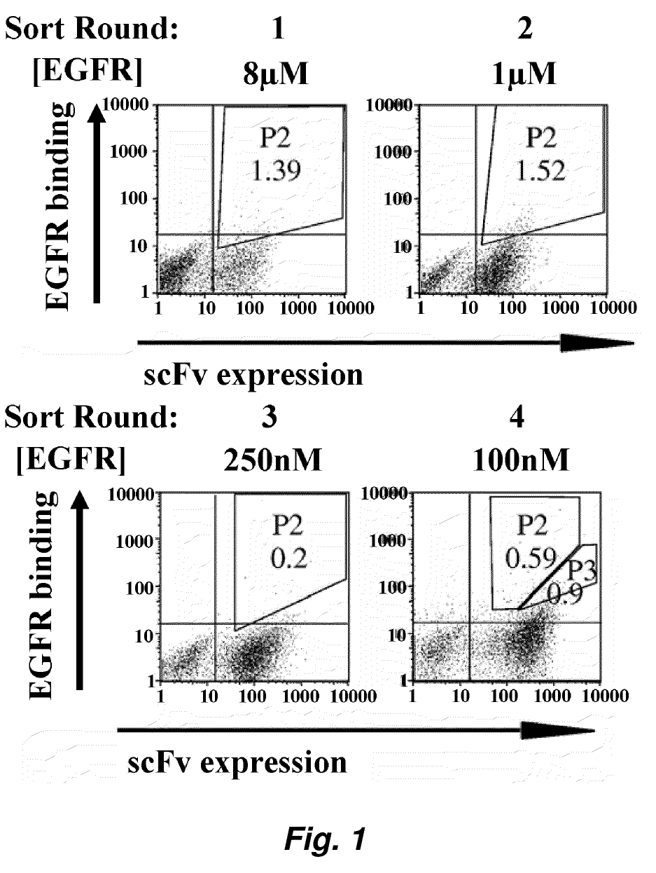

EX₂X₃X₄AX₆X₇EIX₁₀X₁₁LPNLNX₁₇X₁₈QX₂₀X₂₁AFIX₂₅SLX₂₈D

where, independently of each other, X₂ is selected from M, F, V, L, I and S; X₃ is selected from W, D, E and L; X₄ is selected from I, V, G, S, M, L, A, T, N, D and W; X₆ is selected from W, V, L, I, M and S; X₇ is selected from D, E, N and K; X₁₀ is selected from R, G, H and K; X₁₁ is selected from D, N, E, Y and S; X₁₇ is selected from G, W and A; X₁₈ is selected from W, G and A; X₂₀ is selected from M, L, F, A and E; X₂₁ is selected from T, D, N, A and Q; X₂₅ is selected from A, S, N, G and L; and X₂₈ is selected from L, W, V, F and A. Particular illustrative embodiments, include, but are not limited to compounds of the formulas: EMWX₄AWX₇EIRX₁₁ILPNLNGWQMTAFIX₂₅SLLD (SEQ ID NO:27), EX₂X₃X₄AX₆X₇EIX₁₀X₁₁ILPNLNGWQMTAFIASLX₂₈D (SEQ ID NO:28), EX₂X₃X₄AX₆X₇EIGX₁₁ILPNLNWGQX₂₀X₂₁AFIX₂₅SLWD (SEQ ID NO:29), EX₂X₃IAVX₇EIGELPNLNWGQX₂₀DAFINSLWD (SEQ ID NO:30), and the like. One particular sequence is VDNKFNK EQQNAFYEILH LPNLNE QRNAFIQSLKD DPSQ SANLLAEAKKLNDAQAPK (SEQ ID NO:31). In addition, over 300 additional sequences are listed in FIG. 1 of this reference and are incorporated herein by reference. In certain embodiments the binding peptides have a KD of less than 100 μM, preferably less than 10 μM and/or are internalized by the target cell.

B EGFR Ligands.

A number of ligands that bind the EGF receptor are known to those of skill in the art. For example TGF-α and TGF-α mutants are known to bind to EGFR. In addition, compounds such as IRESSA® (gefitinib an anilinoquinazoline with the chemical name 4-Quinazolinamine, N-(3-chloro-4-fluorophenyl)-7-methoxy-6-[3-4-morpholin) propoxy), TARCEVA® (erlotinib), PKI-166 (4-phenethylamino-6-(yderoxyl)phenyl-7H-pyrrolo[2,3-d]pyrimidine), SU-11464, and GW-2016 (N-{3-chloro-4-[(3-fluorobenzyl)oxy]phenyl}-6-[5-({[2-(methylsulfonyl)ethyl]amino}methyl)-2-furyl]-4-quinazolinamine) all bind tightly to wild-type (normal) EGFR.

In addition, for example, U.S. Pat. No. 6,941,229, which is incorporated herein by reference, provides methods of designing compounds able to bind to the EGFR based on the 3-D structure coordinates of the EGF receptor crystal.

C) EGFR Antibodies

A number of antibodies are known that bind to the EGF receptor. For example the antibodies panitumumab and cetuximab (ERBITUX®) have been used in clinical studies (see, e.g., Helbling and Bomer (2007) Annals of Oncology, 18(5):963-964).

1) C10 Mutant Antibodies.

As indicated above, in certain embodiments, this invention provides novel isolated antibodies that specifically bind to an extracellular domain of the epidermal growth factor receptor (EGFR). In various embodiments the antibodies are mutants of the C10 antibody (see, e.g., U.S. Pat. No. 7,332,585 and the PCT application WO 2007/084181A2, which are incorporated herein by reference). Also, in certain embodiments, chimeric moieties comprising an antibody of this invention attached to an “effector” are also provided. Where the effector comprises a second (or more) antibodies, a bispecific (or polyspecific) antibody is provided.

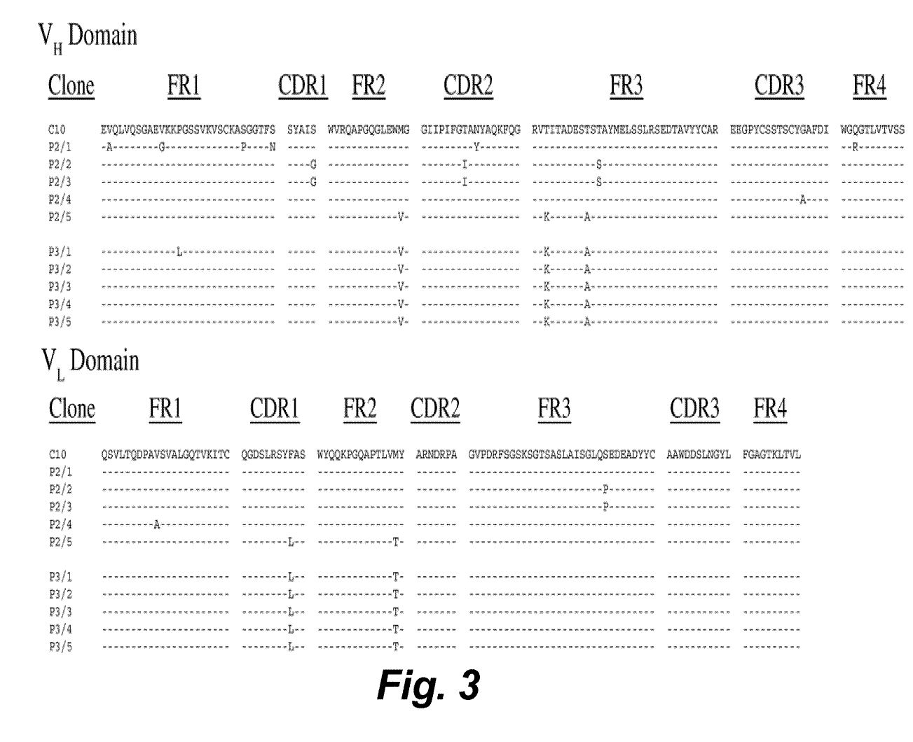

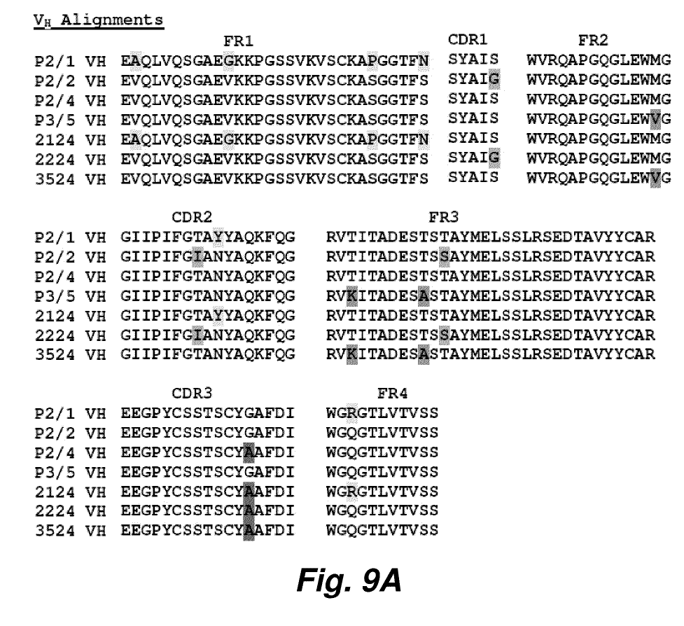

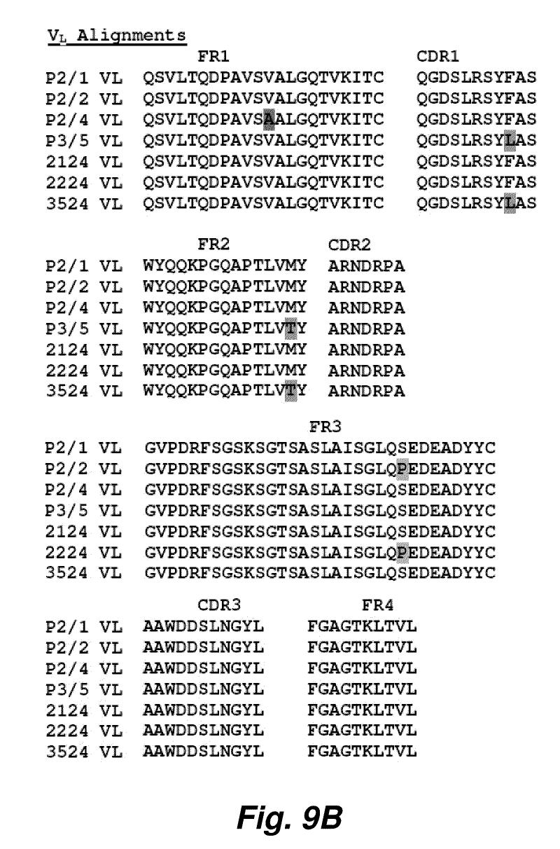

Using phage display approaches, a number of single chain antibodies have been raised that are specific to the epidermal growth factor receptor (EGFR). These single chain Fv antibodies can be used as domains/arms to construct a bispecific or polyspecific antibody, or can be used to create intact (full antibodies, or fragments thereof). The amino acid sequences of various C10 mutants and the various CDRs and framework regions comprising these mutants are illustrated in FIG. 3 and in FIGS. 9A and 9B.

Ten mutant antibodies were produced that have affinity for EGFR that ranges from 3-18 greater affinity (KD=15-88 nM) for EGFR-expressing A431 tumor cells compared to C10 scFv (KD=264 nM). By combining mutations, higher affinity scFv were generated with KD ranging from 0.9 nM to 10 nM. The highest affinity scFv had a 280-fold higher affinity compared to that of the parental C10 scFv.

In various embodiments antibodies are contemplated that comprise one, two, or three variable heavy domain CDRs and/or one, two, or three variable light domain CDRs of one or more of the antibodies shown in FIGS. 3, 9A, and 9B (e.g., P2/1, P2/2, P2/3, P2/4, P2/5, 2124, 2224, 3524, P3/1, P3/2, P3/3, P3/4, and P3/5). In certain embodiments antibodies are contemplated that comprises one, two, or the three VH CDRs and one, two, or the three VL CDRs of one or more of the antibodies shown in FIGS. 3, 9A, and 9B. In certain embodiments the antibody the VH domain and the VL domain of an antibody shown in FIGS. 3, 9A, and 9B. In certain embodiments the antibody is an antibody selected from the group consisting of an intact (full) antibody, an scFv, an IgG, a Fab, an (Fab′)₂, an (scFv′)₂, and the like.

In certain embodiments, these antibodies can be paired with antibodies directed to other epitopes on EGFR or other members of the EGFR family (e.g., C6.5, C6ML3-9, C6 MH3-B1, C6-B1D2, F5, HER3.A5, HER3.F4, HER3.H1, HER3.H3, HER3.E12, HER3.B12, EGFR.E12, EGFR.C10, EGFR.B11, EGFR.E8, HER4.B4, HER4.G4, HER4.F4, HER4.A8, HER4.B6, HER4.D4, HER4.D7, HER4.D11, HER4.D12, HER4.E3, HER4.E7, HER4.F8, HER4.C7 and the like, e.g., as described in U.S. Pat. No. 7,332,585 and the PCT application WO 2007/084181A2 which are both incorporated herein by reference) to form either a bs-scFv antibody with binding specificity for two distinct epitopes on different members of the EGFR protein family or a bs-scFv antibody with binding specificity for two distinct epitopes on the same member of the EGFR protein family.

In certain embodiments, the C10 mutant antibodies are effective to inhibit growth and proliferation of cells expressing high levels of the EGFR (e.g., cancer cells) by themselves. In certain embodiments the C10 mutant antibodies can be attached to an effector and thereby used to preferentially or specifically deliver the effector to cells overexpressing the EGFR receptor (e.g., cancer cells).

2) Identification of Other Antibodies Binding the Same Epitope(s) as Antibodies the Illustrated Anti-EGFR Family Member Antibodies.

The antibodies of this invention need not be limited to the use of the particular antibodies shown in FIGS. 3, 9A, and 9B. In effect, each of these identifies an epitope on the EGFR extracellular domain and these antibodies can readily be used to identify other antibodies that bind to the same epitopes. Thus, in certain embodiments, the antibodies of this invention comprise one or more antibodies that specifically bind an epitope specifically bound by an antibody of FIGS. 3, 9A, and 9B (e.g., an antibody selected from the group consisting of P2/1, P2/2, P2/3, P2/4, P2/5, 2124, 2224, 3524, P3/1, P3/2, P3/3, P3/4, and P3/5).

Such antibodies are readily identified by screening whole antibodies, antibody fragments, or single chain antibodies for their ability to compete with the antibodies listed in FIGS. 3, 9A, and 9B for their ability to bind to EGFR ECD.

In one illustrative embodiment, the antibodies of this invention specifically bind to one or more epitopes recognized by antibodies listed in FIGS. 3, 9A, and 9B. In other words, such antibodies are cross-reactive with one of more of these antibodies. Means of assaying for cross-reactivity are well known to those of skill in the art (see, e.g., Dowbenko et al. (1988) J. Virol. 62: 4703-4711).

For example, in certain embodiments, cross-reactivity can be ascertained by providing an EGFR protein on a cell surface or attached to a solid support and assaying the ability of a test antibody to compete with one or more of the antibodies listed in FIGS. 3, 9A, and 9B for binding to the target EGFR. Thus, immunoassays in a competitive binding format are can be used for crossreactivity determinations. For example, in one embodiment, the EGFR protein is immobilized to a solid support. Antibodies to be tested (e.g., generated by selection from a phage-display library, or generated in a whole antibody library) are added to the assay compete with one or more of the antibodies listed in FIGS. 3, 9A, and 9B for binding to the immobilized polypeptide. The ability of test antibodies to compete with the binding of the antibodies of FIGS. 3, 9A, and 9B to the immobilized protein are compared. The percent crossreactivity above proteins can then be calculated, using standard calculations. If the test antibody competes with one or more of the antibodies of FIGS. 3, 9A, and 9B and has a binding affinity comparable to or greater than about 1×10⁻⁸ M, more preferably greater than 1×10⁻⁹, or 1×10⁻¹⁰, or more generally with an affinity equal to or greater than the corresponding (competing) antibody, e.g., of FIGS. 3, 9A, and 9B then the antibody is well suited for use in the present invention.

In one illustrative embodiment, cross-reactivity is performed by using surface plasmon resonance in a BIAcore. In a BIAcore flow cell, the EGFR protein is coupled to a sensor chip. With a typical flow rate of 5 ml/min, a titration of 100 nM to 1 μM antibody is injected over the flow cell surface for about 5 minutes to determine an antibody concentration that results in near saturation of the surface. Epitope mapping or cross-reactivity is then evaluated using pairs of antibodies at concentrations resulting in near saturation and at least 100 RU of antibody bound. The amount of antibody bound is determined for each member of a pair, and then the two antibodies are mixed together to give a final concentration equal to the concentration used for measurements of the individual antibodies. Antibodies recognizing different epitopes show an essentially additive increase in the RU bound when injected together, while antibodies recognizing identical epitopes show only a minimal increase in RU. In a particularly preferred embodiment, antibodies are said to be cross-reactive if, when “injected” together they show an essentially additive increase (preferably an increase by at least a factor of about 1.4, more preferably an increase by at least a factor of about 1.6, and most preferably an increase by at least a factor of about 1.8 or 2.

Cross-reactivity at the epitopes recognized by the antibodies listed in FIGS. 3 and 9 can ascertained by a number of other standard techniques (see, e.g., Geysen et al (1987) J. Immunol. Meth. 102: 259-274).

In addition, number of the antibodies identified FIGS. 3, 9A, and 9B have been sequenced. The amino acid sequences comprising the complementarity determining regions (CDRs) are therefore known. Using this sequence information, the same or similar complementarity determining regions can be engineered into other antibodies to produce chimeric full size antibodies and/or antibody fragments, e.g., to ensure species compatibility, to increase serum half-life, and the like. A large number of methods of generating chimeric antibodies are well known to those of skill in the art (see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369).

B) Phage Display Methods to Select Other “Related” Anti-EGFR Family Member Antibodies.

1) Chain Shuffling Methods.

One approach to creating modified single-chain antibody (scFv) gene repertoires has been to replace the original V_{H} or V_{L} gene with a repertoire of V-genes to create new partners (chain shuffling) (Clackson et al. (1991) Nature. 352: 624-628). Using chain shuffling and phage display, the affinity of a human scFv antibody fragment that bound the hapten phenyloxazolone (phOx) was increased from 300 nM to 1 nM (300 fold) (Marks et al. (1992) Bio/Technology 10: 779-783).

Thus, for example, to alter the affinity of an anti-EGFR antibody (e.g., a C10 mutant), a mutant scFv gene repertoire can be created containing a V_{H} gene of the prototypic antibodies (e.g., as shown in FIGS. 3 and 9A) and a human V_{L} gene repertoire (light chain shuffling). The scFv gene repertoire can be cloned into a phage display vector, e.g., pHEN-1 (Hoogenboom et al. (1991) Nucleic Acids Res., 19: 4133-4137) or other vectors and, after transformation, a library of transformants is obtained.

Similarly, for heavy chain shuffling, the anti-EGFR antibody V_{L} CDR1 and/or CDR2, and/or CDR3 and light chain e.g. as shown in FIGS. 3 and 9B) are cloned into a vector containing a human V_{H} gene repertoire to create a phage antibody library transformants. For detailed descriptions of chain shuffling to increase antibody affinity see Schier et al. (1996) J. Mol. Biol., 255: 28-43, and the like.

2) Site-Directed Mutagenesis to Improve Binding Affinity.

The majority of antigen contacting amino acid side chains are typically located in the complementarity determining regions (CDRs), three in the V_{H} (CDR1, CDR2, and CDR3) and three in the V_{L} (CDR1, CDR2, and CDR3) (Chothia et al. (1987) J. Mol. Biol., 196: 901-917; Chothia et al. (1986) Science, 233: 755-758; Nhan et al. (1991) J. Mol. Biol., 217: 133-151). These residues contribute the majority of binding energetics responsible for antibody affinity for antigen. In other molecules, mutating amino acids which contact ligand has been shown to be an effective means of increasing the affinity of one protein molecule for its binding partner (Lowman et al. (1993) J. Mol. Biol., 234: 564-578; Wells (1990) Biochemistry, 29: 8509-8516). Site-directed mutagenesis of CDRs and screening against the cells overexpressing one or more EGFR family members can produce antibodies having improved binding affinity.

3) CDR Randomization to Produce Higher Affinity Human scFv.

In an extension of simple site-directed mutagenesis, mutant antibody libraries can be created where partial or entire CDRs are randomized (V_{L} CDR1 CDR2 and/or CDR3 and/or V_{H} CDR1, CDR2 and/or CDR3). In one embodiment, each CDR is randomized in a separate library, using a known antibody (e.g., a C10 mutant) as a template. The CDR sequences of the highest affinity mutants from each CDR library are combined to obtain an additive increase in affinity. A similar approach has been used to increase the affinity of human growth hormone (hGH) for the growth hormone receptor over 1500 fold from 3.4×10⁻¹⁰ to 9.0×10⁻¹³ M (Lowman et al. (1993) J. Mol. Biol., 234: 564-578).

V_{H} CDR3 often occupies the center of the binding pocket, and thus mutations in this region are likely to result in an increase in affinity (Clackson et al. (1995) Science, 267: 383-386). In one embodiment, four V_{H} CDR3 residues are randomized at a time using the nucleotides NNS (see, e.g., Schier et al. (1996) Gene, 169: 147-155; Schier and Marks (1996) Human Antibodies and Hybridomas. 7: 97-105, 1996; and Schier et al. (1996) J. Mol. Biol. 263: 551-567).

C) Creation of Other Antibody Forms.

Using the known and/or identified sequences (e.g. V_{H} and/or V_{L} sequences) of the single chain antibodies provided herein other antibody forms can readily be created. Such forms include, but are not limited to multivalent antibodies, full antibodies, scFv, (scFv′)₂, Fab, (Fab′)₂, chimeric antibodies, and the like.

1) Creation of Homodimers.

For example, to create (scFv′)₂ antibodies, two scFvs are joined, either directly, or through a linker (e.g., a carbon linker, a peptide, etc.), or through a disulfide bond between, for example, two cysteins. Thus, for example, to create disulfide linked scFv, a cysteine residue can be introduced by site directed mutagenesis at the carboxy-terminus of the antibodies described herein.

An scFv can be expressed from this construct, purified by IMAC, and analyzed by gel filtration. To produce (scFv′)₂ dimers, the cysteine is reduced by incubation with 1 mM 3-mercaptoethanol, and half of the scFv blocked by the addition of DTNB. Blocked and unblocked scFvs are incubated together to form (scFv′)₂ and the resulting material can be analyzed by gel filtration. The affinity of the resulting dimer can be determined using standard methods, e.g. by BIAcore.

In one illustrative embodiment, the (scFv′)₂ dimer is created by joining the scFv′ fragments through a linker, more preferably through a peptide linker. This can be accomplished by a wide variety of means well known to those of skill in the art. For example, one preferred approach is described by Holliger et al. (1993) Proc. Natl. Acad. Sci. USA, 90: 6444-6448 (see also WO 94/13804).

It is noted that using the V_{H} and/or V_{L} sequences provided herein Fabs and (Fab′)₂ dimers can also readily be prepared. Fab is a light chain joined to V_{H}-C_{H}1 by a disulfide bond and can readily be created using standard methods known to those of skill in the art. The F(ab)′₂ can be produced by dimerizing the Fab, e.g. as described above for the (scFv′)₂ dimer.

2) Chimeric Antibodies.

The antibodies of this invention also include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; Morrison et al. (1984) Proc. Natl. Acad. Sci. 81: 6851-6855, etc.).

While the prototypic antibodies provided herein are fully human antibodies, chimeric antibodies are contemplated, particularly when such antibodies are to be used in species other than humans (e.g., in veterinary applications). Chimeric antibodies are antibodies comprising portions from two different species (e.g. a human and non-human portion). Typically, the antigen combining region (or variable region) of a chimeric antibody is derived from a one species source and the constant region of the chimeric antibody (which confers biological effector function to the immunoglobulin) is derived from another source. A large number of methods of generating chimeric antibodies are well known to those of skill in the art (see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369, and PCT application WO 91/0996).

In general, the procedures used to produce chimeric antibodies consist of the following steps (the order of some steps may be interchanged): (a) identifying and cloning the correct gene segment encoding the antigen binding portion of the antibody molecule; this gene segment (known as the VDJ, variable, diversity and joining regions for heavy chains or VJ, variable, joining regions for light chains, or simply as the V or variable region or V_{H} and V_{L} regions) may be in either the cDNA or genomic form; (b) cloning the gene segments encoding the human constant region or desired part thereof; (c) ligating the variable region to the constant region so that the complete chimeric antibody is encoded in a transcribable and translatable form; (d) ligating this construct into a vector containing a selectable marker and gene control regions such as promoters, enhancers and poly(A) addition signals; (e) amplifying this construct in a host cell (e.g., bacteria); (f) introducing the DNA into eukaryotic cells (transfection) most often mammalian lymphocytes; and culturing the host cell under conditions suitable for expression of the chimeric antibody.

Antibodies of several distinct antigen binding specificities have been manipulated by these protocols to produce chimeric proteins (e.g., anti-TNP: Boulianne et al. (1984) Nature, 312: 643; and anti-tumor antigens: Sahagan et al. (1986) J. Immunol., 137: 1066). Likewise several different effector functions have been achieved by linking new sequences to those encoding the antigen binding region. Some of these include enzymes (Neuberger et al. (1984) Nature 312: 604), immunoglobulin constant regions from another species and constant regions of another immunoglobulin chain (Sharon et al. (1984) Nature 309: 364; Tan et al., (1985) J. Immunol. 135: 3565-3567).

In certain embodiments, a recombinant DNA vector is used to transfect a cell line that produces a cancer specific antibody of this invention. The novel recombinant DNA vector contains a “replacement gene” to replace all or a portion of the gene encoding the immunoglobulin constant region in the cell line (e.g., a replacement gene may encode all or a portion of a constant region of a human immunoglobulin, a specific immunoglobulin class, or an enzyme, a toxin, a biologically active peptide, a growth factor, inhibitor, or a linker peptide to facilitate conjugation to a drug, toxin, or other molecule, etc.), and a “target sequence” that allows for targeted homologous recombination with immunoglobulin sequences within the antibody producing cell.

In another embodiment, a recombinant DNA vector is used to transfect a cell line that produces an antibody having a desired effector function, (e.g., a constant region of a human immunoglobulin) in which case, the replacement gene contained in the recombinant vector may encode all or a portion of a region of an antibody of this invention and the target sequence contained in the recombinant vector allows for homologous recombination and targeted gene modification within the antibody producing cell. In either embodiment, when only a portion of the variable or constant region is replaced, the resulting chimeric antibody can define the same antigen and/or have the same effector function yet be altered or improved so that the chimeric antibody may demonstrate a greater antigen specificity, greater affinity binding constant, increased effector function, or increased secretion and production by the transfected antibody producing cell line, etc.

Regardless of the embodiment practiced, the processes of selection for integrated DNA (via a selectable marker), screening for chimeric antibody production, and cell cloning, can be used to obtain a clone of cells producing the chimeric antibody.

Thus, a piece of DNA that encodes a modification for a monoclonal antibody can be targeted directly to the site of the expressed immunoglobulin gene within a B-cell or hybridoma cell line. DNA constructs for any particular modification can be made to alter the protein product of any monoclonal cell line or hybridoma. The level of expression of chimeric antibody should be higher when the gene is at its natural chromosomal location rather than at a random position. Detailed methods for preparation of chimeric (humanized) antibodies can be found in U.S. Pat. No. 5,482,856.

3) Intact Human Antibodies.

In another embodiment, this invention provides for intact, fully human antibodies. Such antibodies can readily be produced in a manner analogous to making chimeric human antibodies. In this instance, instead of using a recognition function derived, e.g. from a murine, the fully human recognition function (e.g., V_{H} and V_{L}) of the antibodies described herein is utilized.

4) Diabodies.

In certain embodiments, this invention contemplates diabodies comprising one or more of the V_{H} and V_{L} domains described herein. The term “diabodies” refers to antibody fragments typically having two antigen-binding sites. The fragments typically comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Holliger et al. (1993) Proc. Natl. Acad. Sci. USA 90: 6444-6448.

In short, using routine methods, the antibodies listed in FIGS. 3 and 9 can readily be used to generate or identify other antibodies (full length, antibody fragments, single-chain, and the like) that bind to the same epitope. Similarly, the antibodies listed in FIGS. 3 and 9 can readily be utilized to generate other antibodies that have the same or similar complementarity determining regions (CDRs).

### II. Preparation of Antibody or Other Affinity Moieties.

The antibodies, bispecific antibodies, EGFR binding peptides, EGFR affibodies, chimeric moieties, and the like described herein can be made by methods well known to those of skill in the art.

A) Chemical Synthesis.

Using the sequence information provided herein, for example, the antibodies of this invention (e.g., C10 mutants in FIGS. 3 and 9, or variants thereof) can be chemically synthesized using well known methods of peptide synthesis. Solid phase synthesis in which the C-terminal amino acid of the sequence is attached to an insoluble support followed by sequential addition of the remaining amino acids in the sequence is one preferred method for the chemical synthesis of single chain antibodies. Techniques for solid phase synthesis are described by Barany and Merrifield, Solid Phase Peptide Synthesis; pp. 3-284 in The Peptides: Analysis, Synthesis, Biology. Vol. 2: Special Methods in Peptide Synthesis, Part A., Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.

B) Recombinant Expression of Antibodies.

In certain preferred embodiments, the antibodies of this invention e.g., C10 mutants in FIGS. 3 and 9, or variants thereof), and/or bispecific (e.g., multivalent) moieties, etc., are recombinantly expressed using standard techniques well known to those of skill in the art. The methods typically involve preparing a nucleic acid construct that encodes the desired antibody construct, transfecting a cell with the construct, expressing the encoded construct in the cell and then recovering the desired construct.

Using the sequence information provided herein, nucleic acids encoding the desired antibody can be chemically synthesized according to a number of standard methods known to those of skill in the art. Oligonucleotide synthesis, is preferably carried out on commercially available solid phase oligonucleotide synthesis machines (Needham-VanDevanter et al. (1984) Nucleic Acids Res. 12: 6159-6168) or manually synthesized using the solid phase phosphoramidite triester method described by Beaucage et. al. (Beaucage et. al. (1981) Tetrahedron Letts. 22(20): 1859-1862). Alternatively, nucleic acids encoding the antibody can be amplified and/or cloned according to standard methods.

Molecular cloning techniques to achieve these ends are known in the art. A wide variety of cloning and in vitro amplification methods are suitable for the construction of recombinant nucleic acids. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel). Methods of producing recombinant immunoglobulins are also known in the art. See, Cabilly, U.S. Pat. No. 4,816,567; and Queen et al. (1989) Proc. Natl. Acad. Sci. USA 86: 10029-10033. In addition, detailed protocols for the expression of antibodies are also provided by Liu et al. (2004) Cancer Res. 64: 704-710, Poul et al. (2000) J. Mol. Biol. 301: 1149-1161, and the like. In addition, the expression of illustrative scFv are described herein in Example 1.

### III. Immunoconjugates/Chimeric Moieties.

In many embodiments, the C10 mutant antibodies described herein are capable of inhibiting cancer cell growth and/or proliferation without the use of any additional “effector”. However, in certain embodiments, the antibodies or other EGFR affinity moieties are additionally coupled to an effector which can, optionally comprise a microparticle or nanoparticle thereby forming chimeric moieties that preferentially target/deliver the effector to a cell overexpressing the EGFR. When contacted to the target cell under conditions permitting endocytosis, the chimeric moiety is typically internalized by the target cell.

It was discovered that in certain embodiments, the attachment of multiple EGFR affinity moieties (e.g., C10 mutant antibodies, affibodies, etc.) to an effector (e.g., a nanoparticle) can significantly enhance internalization into a cell expressing the EGFR Moreover, surprisingly, it was discovered that where certain density of affinity moieties per nanoparticle is maintained it is possible to achieve a high level of internalization even with lower affinity moieties even in the cells with moderate number of EGFR on their surface (less than 500,000), such as MDAMB231 human carcinoma cells. Thus, for example, affinity moieties having a Kd for EGFR of about 100 nM or more/for example about 264 nM (comparable to C10 Ab) coupled to a liposome composed of 80,000 phospholipid molecules (total surface area about 24,000 nm²) show effective internalization at greater than about 50 or 74 moieties per liposome, more preferably greater than about 100 moieties per liposome, and most preferably greater than about 148, 150, 175, or 200 moieties per liposome. Affinity moieties having a Kd for EGFR of about 10 nM or more, such as about 15 nM, attached to a similar liposome showed effective internalization at a density of greater than about 25 moieties per liposome, preferably at greater than 35, 37 or 50 moieties per liposome and more preferably greater than about 74, 75, 80, 90, or 100 moieties per liposome. Affinity moieties having a Kd for EGFR of about 0.94 nM attached to a liposome showed effective internalization at a density of greater than about 12 moieties per liposome, preferably greater than about 20 or 25 moieties per liposome, and more preferably 15 greater than 30, 35, 37, 50, or 70 moieties per 24,000 nm² of the liposome surface (see, e.g., Table 1).

TABLE 1
Illustrative densities of EGFR affinity moieties
(copies per 24,000 nm² of the liposome surface)
to achieve effective internalization.

| | |
|---|---|
| | Copies of |
| Kd for | antibody |
| EGFR | per liposome |

| | | | |
|---|---|---|---|
| 263 | nmol | ≧74 | (preferred ≧ 148) |
| 15 | nmol | ≧25 | (preferred ≧ 37) |
| 0.94 | nmol | ≧12 | (preferred ≧ 25) |

Accordingly in certain embodiments, compositions are contemplated comprising a plurality of affinity moieties (e.g., antibodies) attached to an effector (e.g., a microparticle, a nanoparticle, and the like). Where the effector comprises a microparticle or nanoparticle, active agents (e.g., the agent whose activity is to be delivered to the cell) can be contained within the particle, admixed with the particle, or attached to the surface of the particle. Such active agents include, but are not limited to imaging compositions, radiosensitizers, cytotoxins, therapeutic drugs, antisense molecules, siRNA, and the like. In various embodiments the active agent(s) can be coupled directly to one or more affinity moieties to produce a chimeric molecule and thereby omit the microparticle or nanoparticle.

A) Lipidic Microparticles or Nanoparticles.

In certain embodiments, the microparticles or nanoparticles are lipidic particles. Lipidic particles are microparticles or nanoparticles that include at least one lipid component forming a condensed lipid phase. Typically, a lipidic nanoparticle has preponderance of lipids in its composition. The exemplary condensed lipid phases are solid amorphous or true crystalline phases; isomorphic liquid phases (droplets); and various hydrated mesomorphic oriented lipid phases such as liquid crystalline and pseudocrystalline bilayer phases (L-alpha, L-beta, P-beta, Lc), interdigitated bilayer phases, and nonlamellar phases (inverted hexagonal H-I, H-II, cubic Pn3m) (see The Structure of Biological Membranes, ed. by P. Yeagle, CRC Press, Bora Raton, Fla., 1991, in particular ch. 1-5, incorporated herein by reference.). Lipidic microparticles include, but are not limited to a liposome, a lipid-nucleic acid complex, a lipid-drug complex, a solid lipid particle, and a microemulsion droplet. Methods of making and using these types of lipidic microparticles and nanoparticles, as well as attachment of affinity moieties, e.g., antibodies, to them are known in the art (see, e.g., U.S. patents: U.S. Pat. Nos. 5,077,057; 5,100,591; 5,616,334; 6,406,713 (drug-lipid complexes); U.S. patents: U.S. Pat. Nos. 5,576,016; 6,248,363; Bondi et al. (2003) Drug Delivery 10: 245-250; Pedersen et al. (2006) Eur. J. Pharm. Biopharm. 62: 155-162 (solid lipid particles); U.S. patents: U.S. Pat. Nos. 5,534,502; 6,720,001; Shiokawa et al. (2005) Clin. Cancer Res. 11: 2018-2025 (microemulsions); U.S. Pat. No. 6,071,533 (lipid-nucleic acid complexes)).

A liposome is generally defined as a particle comprising one or more lipid bilayers enclosing an interior, typically an aqueous interior. Thus, a liposome is often a vesicle formed by a bilayer lipid membrane. There are many methods for the preparation of liposomes. Some of them are used to prepare small vesicles (d<0.05 micrometer), some for larger vesicles (d>0.05 micrometer). Some are used to prepare multilamellar vesicles, some for unilamellar ones. In certain embodiments for the present invention, unilamellar vesicles are preferred because a lytic event on the membrane means the lysis of the entire vesicle. However, multilamellar vesicles can also be used, perhaps with reduced efficiency. Methods for liposome preparation are exhaustively described in several review articles such as Szoka and Papahadjopoulos (1980) Ann. Rev. Biophys. Bioeng., 9: 467, Deamer and Uster (1983) Pp. 27-51 In: Liposomes, ed. M. J. Ostro, Marcel Dekker, New York, and the like.

In various embodiments, liposomes of the invention are composed of vesicle-forming lipids, generally including amphipathic lipids having both hydrophobic tail groups and polar head groups. A characteristic of a vesicle-forming lipid is its ability to either (a) form spontaneously into bilayer vesicles in water, as exemplified by the phospholipids, or (b) be stably incorporated into lipid bilayers, by having the hydrophobic portion in contact with the interior, hydrophobic region of the bilayer membrane, and the polar head group oriented toward the exterior, polar surface of the membrane. A vesicle-forming lipid for use in the present invention is any conventional lipid possessing one of the characteristics described above.

In certain embodiments the vesicle-forming lipids of this type are preferably those having two hydrocarbon tails or chains, typically acyl groups, and a polar head group. Included in this class are the phospholipids, such as phosphatidylcholine (PC), phosphatidylethanolamine (PE), phosphatidic acid (PA), phosphatidylglycerol (PG), and phosphatidylinositol (PI), where the two hydrocarbon chains are typically between about 14-22 carbon atoms in length, and have varying degrees of unsaturation. In certain embodiments preferred phospholipids include PE and PC. One illustrative PC is hydrogenated soy phosphatidylcholine (HSPC). Single chain lipids, such as sphingomyelin (SM), and the like can also be used.

The above-described lipids and phospholipids whose acyl chains have a variety of degrees of saturation can be obtained commercially, or prepared according to published methods. Other lipids that can be included in certain embodiments are sphingolipids and glycolipids. The term “sphingolipid” as used herein encompasses lipids having two hydrocarbon chains, one of which is the hydrocarbon chain of sphingosine. The term “glycolipids” refers to shingolipids comprising also one or more sugar residues.

Lipids for use in the lipidic microparticles or nanoparticles of the present invention can include relatively “fluid” lipids, meaning that the lipid phase has a relatively low lipid melting temperature, e.g., at or below room temperature, or alternately, relatively “rigid” lipids, meaning that the lipid has a relatively high melting point, e.g., at temperatures up to 50° C. As a general rule, the more rigid, i.e., saturated lipids, contribute to greater membrane rigidity in the lipid bilayer structure, and thus to more stable drug retention after active drug loading. In certain embodiments preferred lipids of this type are those having phase transition temperatures above about 37° C.

In various embodiments the liposomes may additionally include lipids that can stabilize a vesicle or liposome composed predominantly of phospholipids. An illustrative lipids of this group is cholesterol at levels between 25 to 45 mole percent.

In certain embodiments liposomes used in the invention contain between 30-75 percent phospholipids, e.g., phosphatidylcholine (PC), 25-45 percent cholesterol. One illustrative liposome formulation contains 60 mole percent phosphatidylcholine and 40 mole percent cholesterol.

In various embodiments the liposomes of the invention include a surface coating of a hydrophilic polymer chain. “Surface-coating” refers to the coating of any hydrophilic polymer on the surface of liposomes. The hydrophilic polymer is included in the liposome by including in the liposome composition one or more vesicle-forming lipids derivatized with a hydrophilic polymer chain. The vesicle-forming lipids which can be used are any of those described above for the first vesicle-forming lipid component, however, in certain embodiments, vesicle-forming lipids with diacyl chains, such as phospholipids, are preferred. One illustrative phospholipid is phosphatidylethanolamine (PE), which contains a reactive amino group convenient for coupling to the activated polymers. One illustrative PE is distearoyl PE (DSPE). Another example is non-phospholipid double chain amphiphilic lipids, such as diacyl- or dialkylglycerols, derivatized with a hydrophilic polymer chain.

In certain embodiments a hydrophilic polymer for use in coupling to a vesicle forming lipid is polyethyleneglycol (PEG), preferably as a PEG chain having a molecular weight between 1,000-10,000 Daltons, more preferably between 1,000-5,000 Daltons, most preferably between 2,000-5,000 Daltons. Methoxy or ethoxy-capped analogues of PEG are also useful hydrophilic polymers, commercially available in a variety of polymer sizes, e.g., 120-20,000 Daltons.

Other hydrophilic polymers that can be suitable include, but are not limited to polylactic acid, polyglycolic acid, polyvinylpyrrolidone, polymethyloxazoline, polyethyloxazoline, polyhydroxypropyl methacrylamide, polymethacrylamide, polydimethylacrylamide, and derivatized celluloses, such as hydroxymethylcellulose or hydroxyethylcellulose.

Preparation of lipid-polymer conjugates containing these polymers attached to a suitable lipid, such as PE, have been described, for example in U.S. Pat. No. 5,395,619, which is expressly incorporated herein by reference, and by Zalipsky in STEALTH LIPOSOMES (1995). In certain embodiments, typically, between about 1-20 mole percent of the polymer-derivatized lipid is included in the liposome-forming components during liposome formation. Polymer-derivatized lipids suitable for practicing the invention are also commercially available (e.g. SUNBRITE®, NOF Corporation, Japan.).

In various embodiments the hydrophilic polymer chains provide a surface coating of hydrophilic chains sufficient to extend the blood circulation time of the liposomes in the absence of such a coating. The extent of enhancement of blood circulation time is severalfold over that achieved in the absence of the polymer coating, as described in U.S. Pat. No. 5,013,556, which is expressly incorporated herein by reference.

The liposomes may be prepared by a variety of techniques, such as those detailed in Szoka et al. (1980) Ann. Rev. Biophys. Bioeng. 9: 467, and a specific example of liposomes prepared in support of the present invention is set forth in Example 1. In certain embodiments the liposomes are multilamellar vesicles (MLVs), which can be formed by simple lipid-film hydration techniques. In this procedure, a mixture of liposome-forming lipids and including a vesicle-forming lipid derivatized with a hydrophilic polymer are dissolved in a suitable organic solvent which is evaporated in a vessel to form a dried thin film. The film is then covered by an aqueous medium to form MLVs, typically with sizes between about 0.1 to 10 microns. Illustrative methods of preparing derivatized lipids and of forming polymer-coated liposomes have been described in U.S. Pat. Nos. 5,013,556, 5,631,018 and 5,395,619, which are incorporated herein by reference.

After liposome formation, the vesicles may be sized to achieve a size distribution of liposomes within a selected range, according to known methods. In certain embodiments the liposomes are uniformly sized to a selected size range between 0.04 to 0.25 μm. Small unilamellar vesicles (SUVs), typically in the 0.04 to 0.08 μm range, can be prepared by extensive sonication or homogenization of the liposomes. Homogeneously sized liposomes having sizes in a selected range between about 0.08 to 0.4 microns can be produced, e.g., by extrusion through polycarbonate membranes or other defined pore size membranes having selected uniform pore sizes ranging from 0.03 to 0.5 microns, typically, 0.05, 0.08, 0.1, or 0.2 microns. The pore size of the membrane corresponds roughly to the largest size of liposomes produced by extrusion through that membrane, particularly where the preparation is extruded two or more times through the same membrane. The sizing is typically carried out in the original lipid-hydrating buffer, so that the liposome interior spaces retain this medium throughout the initial liposome processing steps.

In certain embodiments the liposomes are prepared to include an ion gradient, such as a pH gradient or an ammonium or amine ion gradient, across the liposome lipid bilayer in order to effect loading of the liposomes with a substance of interest, e.g., a pharmaceutical (drug). A liposome may also contain substances, such as polyvalent ions, reducing the rate of drug escape from the liposome. One method for preparing such liposomes loaded with a drug is set forth in U.S. Patent Publication 2007/0116753 which is incorporated herein by reference.

In one illustrative approach a mixture of liposome-forming lipids is dissolved in a suitable organic solvent and evaporated in a vessel to form a thin film. The film is then covered with an aqueous medium containing the solute species that will form the aqueous phase in the liposome interior spaces in the final liposome preparation. The lipid film hydrates to form multi-lamellar vesicles (MLVs), typically with heterogeneous sizes between about 0.1 to 10 microns. The liposome are then sized, as described above, to a uniform selected size range.

After sizing, the external medium of the liposomes is treated to produce an ion gradient across the liposome membrane, which is typically a lower inside/higher outside concentration gradient. This may be done in a variety of ways, e.g., by (i) diluting the external medium, (ii) dialysis against the desired final medium, (iii) molecular-sieve chromatography, e.g., using SEPHADEX G-50, against the desired medium, or (iv) high-speed centrifugation and resuspension of pelleted liposomes in the desired final medium. The external medium which is selected will depend on the mechanism of gradient formation and the external pH desired, as will now be considered.

In one approach for generating a pH gradient, the hydrated sized liposomes have a selected internal-medium pH. The suspension of the liposomes is titrated until a desired final pH is reached, or treated as above to exchange the external phase buffer with one having the desired external pH. For example, the original medium may have a pH of 5.5, in a selected buffer, e.g., glutamate or phosphate buffer, and the final external medium may have a pH of 8.5 in the same or different buffer. The internal and external media are preferably selected to contain about the same osmolarity, e.g., by suitable adjustment of the concentration of buffer, salt, or low molecular weight solute, such as sucrose.

In another approach, the proton gradient used for drug loading is produced by creating an ammonium ion gradient across the liposome membrane, as described, for example, in U.S. Pat. No. 5,192,549. Here the liposomes are prepared in an aqueous buffer containing an ammonium salt, typically 0.1 to 0.3 M ammonium salt, such as ammonium sulfate, at a suitable pH, e.g., 5.5 to 7.5. After liposome formation and sizing, the external medium is exchanged for one lacking ammonium ions, e.g., the same buffer but one in which ammonium sulfate is replaced by NaCl or a sugar that gives the same osmolarity inside and outside of the liposomes.

After liposome formation, the ammonium ions inside the liposomes are in equilibrium with ammonia and protons. Ammonia is able to penetrate the liposome bilayer and escape from the liposome interior. Escape of ammonia continuously shifts the equilibrium within the liposome toward the right, to production of protons.

While the foregoing discussion pertains to the formation of liposomes, similar lipids and lipid compositions can be used to form other lipidic microparticles or nanoparticles such as a solid lipid particle, a microemulsion, and the like.

Further, the liposomes may be prepared for attachment to EGFR affinity moieties (e.g. C10 mutant antibodies). Here the lipid component included in the liposomes would include either a lipid derivatized with the affinity moiety, or a lipid having a polar-head chemical group, e.g., on a linker, that can be derivatized with the targeting molecule in preformed liposomes, according to known methods.

Methods of functionalizing lipids and liposomes with affinity moieties such as antibodies are well known to those of skill in the art (see, e.g., DE 3,218,121; Epstein et al. (1985) Proc. Natl. Acad. Sci., USA, 82:3688 (1985); Hwang et al. (1980) Proc. Natl. Acad. Sci., USA, 77: 4030; EP 52,322; EP 36,676; EP 88,046; EP 143,949; EP 142,641; Japanese patent application 83-118008; U.S. Pat. Nos. 4,485,045 and 4,544,545; and EP 102,324, all of which are incorporated herein by reference). One illustrative method for attachment of proteinaceous affinity moieties to lipidic microparticles is described in U.S. Pat. No. 6,210,707, incorporated herein by reference. In addition, formulation of immunoliposomes is illustrated herein in Example 1.

B) Polymeric Microparticles or Nanoparticles and Micelles.

Microparticle and especially nanoparticle-based drug delivery systems have considerable potential for treatment of various pathologies. Technological advantages of polymeric microparticles or nanoparticles used as drug carriers are high stability, high carrier capacity, feasibility of incorporation of both hydrophilic and hydrophobic substances, and feasibility of variable routes of administration, including oral application and inhalation. Polymeric nanoparticles can also be designed to allow controlled (sustained) drug release from the matrix. These properties of nanoparticles enable improvement of drug bioavailability and reduction of the dosing frequency.

Polymeric nanoparticles are typically micron or submicron (<1 μm) colloidal particles. This definition includes monolithic nanoparticles (nanospheres) in which the drug is adsorbed, dissolved, or dispersed throughout the matrix and nanocapsules in which the drug is confined to an aqueous or oily core surrounded by a shell-like wall. Alternatively, in certain embodiments, the drug can be covalently attached to the surface or into the matrix.

Polymeric microparticles and nanoparticles are typically made from biocompatible and biodegradable materials such as polymers, either natural (e.g., gelatin, albumin) or synthetic (e.g., polylactides, polyalkylcyanoacrylates), or solid lipids. In the body, the drug loaded in nanoparticles is usually released from the matrix by diffusion, swelling, erosion, or degradation. One commonly used material is poly(lactide-co-glycolide) (PLG).

Methods of fabricating and loading polymeric nanoparticles or microparticles are well known to those of skill in the art. Thus, for example, Matsumoto et al. (1999) Intl. J. Pharmaceutics, 185: 93-101 teaches the fabrication of poly(L-lactide)-poly(ethylene glycol)-poly(L-lactide) nanoparticles, Chawla et al. (2002) Intl. J. Pharmaceutics 249: 127-138, teaches the fabrication and use of poly(ε-caprolactone) nanoparticles delivery of tamifoxen, and Bodmeier et al. (1988) Intl. J. Pharmaceutics, 43: 179-186, teaches the preparation of poly(D,L-lactide) microspheres using a solvent evaporation method.” Intl. J. Pharmaceutics, 1988, 43, 179-186. Other nanoparticle formulations are described, for example, by Williams et al. (2003) J. Controlled Release, 91: 167-172; Leroux et al. (1996) J. Controlled Release, 39: 339-350; Soppimath et al. (2001) J. Controlled Release, 70:1-20; Brannon-Peppas (1995) Intl. J. Pharmaceutics, 116: 1-9; and the like.

Another kind of nanoparticle suitable for practicing the instant invention is a micelle. As used herein, a “micelle” refers to an aggregate of amphiphilic molecules in an aqueous medium, having an interior core and an exterior surface, wherein the amphiphilic molecules are predominantly oriented with their hydrophobic portions forming the core and hydrophilic portions forming the exterior surface. Micelles are typically in a dynamic equilibrium with the amphiphilic molecules or ions from which they are formed existing in solution in a non-aggregated form. Many amphiphilic compounds, including in particular. detergents, surfactants, amphiphilic polymers, lipopolymers (such as PEG-lipids), bile salts, single-chain phospholipids and other single-chain amphiphiles, and amphipathic pharmaceutical compounds are known to spontaneously form micelles in aqueous media above certain concentration, known as critical micellization concentration, or CMC. Unlike lipidic microparticles and nanoparticles, amphipathic, e.g., lipid, components of a micelle, as defined herein, do not form bilayer phases, nonbilayer mesophases, isotropic liquid phases or solid amorphous or crystalline phases. The concept of a micelle, as well as the methods and conditions for their formation, are well known to skilled in the art. Micelles can co-exist in solution with lipidic microparticles and nanoparticles (see, e.g., Liposome Technology, Third Edition, vol. 1, ch. 11, p. 209-239, Informa, London, 2007). Micelles are useful in carrying and targeting pharmaceutical agents. The uses of micelles as carriers for pharmaceuticals as well as the methods of making pharmaceutical micelles and attachment to micelles of moieties having affinity to target cells and/or tissues, including affinity moieties binding to EGFR, are known in the art (see, e.g., Torchilin (2007) Pharmaceutical Res. 24: 1-16; Lukyanov and Torchilin (2004) Adv. Drug Delivery Reviews 56: 1273-1289; Torchilin et al. (2003) Proc. Natl. Acad. Sci., USA, 100: 6039-6044; Zeng et al. (2006) Bioconjugate Chemistry 17: 399-409; Sutton et al. (2007) Pharmaceutical Research 24: 1029-1046; Lee et al. (2007) Molecular Pharmacology, 4: 769-781, all incorporated herein by reference).

C) Chimeric Molecules.

In certain embodiments, the microparticle or nanoparticle is absent and the EGFR affinity moiety is attached directly or through a linker to an active agent thereby forming a chimeric molecule. A chimeric molecule refers to a molecule or composition wherein two or more molecules that exist separately in their native state are joined together to form a single molecule moiety or composition having the desired functionality of its constituent members. Typically, one of the constituent molecules of a chimeric moiety is a “targeting molecule”, e.g., an anti-EGFR antibody or other affinity moiety that binds EGFR.

Illustrative chimeric molecules in clued one or more EGFR affinity moieties joined to a detectable label, a radiosensitizer, a ligand, a chelate, a cytotoxin, and the like. In certain embodiments one or more EGFR affinity moieties are attached to a second antibody (e.g., an antibody that binds another EGFR epitope, or a different member of the EGFR family) thereby forming a bispecific or polyspecific antibody.

D) Attachment of the Affinity Moieties to the Nanoparticles, Microparticles and/or Active Agent.

The affinity moieties (e.g., C10 mutant antibodies) can be attached to the microparticles or nanoparticles and/or active agent(s) by any of a number of methods known to those of skill in the art. Typically the effector moiety (microparticle, nanoparticle and/or active agent) is conjugated, either directly or through a linker (spacer), to one or more affinity moieties. However, where a chimeric molecule is produced where the affinity moiety is a single chain protein and the active agent is also a protein, it is preferable to recombinantly express the chimeric molecule as a single-chain fusion protein.

1) Conjugation of the Effector Molecule to the Targeting Molecule.

In one illustrative embodiment, the EGFR affinity moieties are chemically conjugated to the effector moiety. Means of chemically conjugating molecules are well known to those of skill.

The procedure for attaching an agent to an antibody or other targeting molecule will vary according to the chemical structure of the agent. Polypeptides typically contain variety of functional groups; e.g., carboxylic acid (COOH) or free amine (—NH₂) groups, which are available for reaction with a suitable functional group on an effector molecule to bind the effector thereto.

Alternatively, the affinity moiety antibody and/or effector moiety can be derivatized to expose or attach additional reactive functional groups. The derivatization can involve attachment of any of a number of linker molecules such as those available from Pierce Chemical Company, Rockford Ill.

A “linker”, as used herein, is a molecule that is used to join the targeting molecule to the effector molecule. The linker is capable of forming covalent bonds to both the targeting molecule and to the effector molecule. Suitable linkers are well known to those of skill in the art and include, but are not limited to, straight or branched-chain carbon linkers, heterocyclic carbon linkers, or peptide linkers. Where an affinity moiety and the effector moiety are or comprise polypeptides, the linkers can be joined to the constituent amino acids through their side groups (e.g., through a disulfide linkage to cysteine). However, in certain embodiments, the linkers will be joined to the alpha carbon amino and carboxyl groups of the terminal amino acids.

A bifunctional linker having one functional group reactive with a group on an effector moiety, and another group reactive with an EGFR affinity moiety, can be used to form the desired conjugate. Alternatively, derivatization can involve chemical treatment of the affinity moiety, e.g., glycol cleavage of a sugar moiety of a glycoprotein antibody with periodate to generate free aldehyde groups. The free aldehyde groups on the antibody can be reacted with free amine or hydrazine groups on an agent to bind the agent thereto. (See U.S. Pat. No. 4,671,958). Procedures for generation of free sulfhydryl groups on polypeptide, such as antibodies or antibody fragments, are also known (See U.S. Pat. No. 4,659,839).

Many procedures and linker molecules for attachment of various compounds including lipids, radionuclide metal chelates, toxins and drugs to proteins such as antibodies are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075). In particular, production of various immunotoxins is well-known within the art and can be found, for example in “Monoclonal Antibody-Toxin Conjugates: Aiming the Magic Bullet,” Thorpe et al., Monoclonal Antibodies in Clinical Medicine, Academic Press, pp. 168-190 (1982), Waldmann (1991) Science, 252: 1657, U.S. Pat. Nos. 4,545,985 and 4,894,443.

In some circumstances, it is desirable to free the effector moiety from the affinity moiety (e.g., C10 mutant antibody) when the chimeric moiety has reached its target site. Therefore, chimeric conjugates comprising linkages that are cleavable in the vicinity of the target site can be used when the effector is to be released at the target site. Cleaving of the linkage to release the agent from the antibody may be prompted by enzymatic activity or conditions to which the immunoconjugate is subjected either inside the target cell or in the vicinity of the target site. When the target site is a tumor, a linker which is cleavable under conditions present at the tumor site (e.g., when exposed to tumor-associated enzymes or acidic pH) may be used.

A number of different cleavable linkers are known to those of skill in the art (see, e.g., U.S. Pat. Nos. 4,618,492; 4,542,225, and 4,625,014). The mechanisms for release of an agent from these linker groups include, for example, irradiation of a photolabile bond and acid-catalyzed hydrolysis. U.S. Pat. No. 4,671,958, for example, includes a description of immunoconjugates comprising linkers that are cleaved at the target site in vivo by the proteolytic enzymes of the patient's complement system. In view of the large number of methods that have been reported for attaching a variety of radiodiagnostic compounds, radiotherapeutic compounds, drugs, toxins, and other agents to antibodies one skilled in the art will be able to determine a suitable method for attaching a given agent to an antibody or other polypeptide.

2 Conjugation of Chelates.

In certain preferred embodiments, the effector moiety comprises a chelate that is attached to an antibody or to an epitope tag. The affinity moiety (e.g., C10 mutant antibody) bears a corresponding epitope tag or antibody so that simple contacting of antibody to the chelate results in attachment of the antibody to the effector. The combining step can be performed after the moiety is used (pretargeting strategy) or the target tissue can be bound to the affinity moiety (e.g., antibody) before the chelate is delivered. Methods of producing chelates suitable for coupling to various targeting moieties are well known to those of skill in the art (see, e.g., U.S. Pat. Nos. 6,190,923, 6,187,285, 6,183,721, 6,177,562, 6,159,445, 6,153,775, 6,149,890, 6,143,276, 6,143,274, 6,139,819, 6,132,764, 6,123,923, 6,123,921, 6,120,768, 6,120,751, 6,117,412, 6,106,866, 6,096,290, 6,093,382, 6,090,800, 6,090,408, 6,088,613, 6,077,499, 6,075,010, 6,071,494, 6,071,490, 6,060,040, 6,056,939, 6,051,207, 6,048,979, 6,045,821, 6,045,775, 6,030,840, 6,028,066, 6,022,966, 6,022,523, 6,022,522, 6,017,522, 6,015,897, 6,010,682, 6,010,681, 6,004,533, and 6,001,329).

3) Production of Fusion Proteins.

Where antibody and the active agent molecule are both single chain proteins and relatively short (i.e., less than about 50 amino acids) they can be synthesized using standard chemical peptide synthesis techniques. Where both components are relatively short the chimeric moiety can be synthesized as a single contiguous polypeptide. Alternatively the antibody and the active agent can be synthesized separately and then fused by condensation of the amino terminus of one molecule with the carboxyl terminus of the other molecule thereby forming a peptide bond. Alternatively, the antibody and effector agent molecules can each be condensed with one end of a peptide spacer molecule thereby forming a contiguous fusion protein.

Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85: 2149-2156 (1963), and Stewart et al., Solid Phase Peptide Synthesis, 2nd ed. Pierce Chem. Co., Rockford, Ill. (1984).

In one embodiment, the where the affinity moiety is a single chain polypeptide and the active agent is a polypeptide, chimeric fusion proteins of the present invention are synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein.

DNA encoding the fusion proteins (e.g., C10 mutant Ab—second Ab) of this invention can be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences can be obtained by the ligation of shorter sequences.

Alternatively, subsequences can be cloned and the appropriate subsequences cleaved using appropriate restriction enzymes. The fragments can then be ligated to produce the desired DNA sequence.

While the two molecules can be essentially directly joined together, or the molecules may be separated by a peptide spacer consisting of one or more amino acids. Generally the spacer will have no specific biological activity other than to join the proteins or to preserve some minimum distance or other spatial relationship between them. However, the constituent amino acids of the spacer can be selected to influence some property of the molecule such as the folding, net charge, or hydrophobicity.

The nucleic acid sequences encoding the fusion proteins can be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion proteins can be purified according to standard procedures of the art, including ammonium sulfate precipitation, affinity columns, column chromatography, gel electrophoresis and the like (see, generally, R. Scopes (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

One of skill in the art would recognize that after chemical synthesis, biological expression, or purification, the EGFR targeted fusion protein can possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem., 268: 14065-14070; Kreitman and Pastan (1993) Bioconjug. Chem., 4: 581-585; and Buchner, et al. (1992) Anal. Biochem., 205: 263-270).

One of skill would recognize that modifications can be made to the fusion proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids placed on either terminus to create conveniently located restriction sites or termination codons.

E) Certain Active Agents/Effectors.

In certain embodiments this invention provides chimeric moieties comprising a microparticle or nanoparticle attached to one, or a plurality of EGFR affinity moieties (e.g., C10 mutant antibodies). In various embodiments the microparticles or nanoparticles can have an active agent (the agent whose activity is to be delivered to the cell) contained within the particle, admixed with the particle, covalently coupled to the particle material, or adsorbed or covalently coupled to the surface of the microparticle. In certain embodiments the microparticle and/or nanoparticle is omitted and the affinity moieties are coupled directly or through a linker to an active agent.

Illustrative active agents include, but are not limited to imaging compositions, radiosensitizers, ligands, chelates, cytotoxins, pharmaceuticals, ribozymes, antisense molecules, RNAi moieties, and the like.

1) Imaging Compositions.

In certain embodiments, the chimeric moieties of this invention can be used to direct detectable labels to a tumor site. This can facilitate tumor detection and/or localization. In certain embodiments, the effector component of the chimeric moiety comprises a “radiopaque” label (e.g., a label that can be easily visualized using x-rays) attached directly to the affinity moiety or associated with a microparticle or nanoparticle. Radiopaque materials are well known to those of skill in the art. The most common radiopaque materials include iodide, bromide or barium salts. Other radiopaque materials are also known and include, but are not limited to organic bismuth derivatives (see, e.g., U.S. Pat. No. 5,939,045), radiopaque polyurethanes (see U.S. Pat. No. 5,346,9810, organobismuth composites (see, e.g., U.S. Pat. No. 5,256,334), radio-opaque barium polymer complexes (see, e.g., U.S. Pat. No. 4,866,132), and the like.

The EGFR affinity moieties can be coupled directly to the radiopaque moiety or they can be attached to, admixed with or contained within a “package” (e.g., a chelate, a liposome, a polymer microbead/nanoparticle, etc.) carrying or containing the radiopaque material as described below.

In addition to radioopaque labels, other labels are also suitable for use in this invention. Detectable labels suitable for use as the effector molecule component of the chimeric molecules of this invention include any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

Among the radionuclides and labels useful in the radionuclide-chelator—(e.g., biotin) conjugates of the present invention, gamma-emitters, positron-emitters, x-ray emitters and fluorescence-emitters are suitable for localization, diagnosis and/or staging, and/or therapy, while beta and alpha-emitters and electron and neutron-capturing agents, such as boron and uranium, also can be used for therapy.

The detectable labels can be used in conjunction with an external detector and/or an internal detector and provide a means of effectively localizing and/or visualizing, e.g., cancer cells overexpressing EGF receptors. Such detection/visualization can be useful in various contexts including, but not limited to pre-operative and intraoperative settings. Thus, in certain embodiment this invention relates to a method of intraoperatively detecting and locating tissues having EGFR markers in the body of a mammal. These methods typically involve administering to the mammal a composition comprising, in a quantity sufficient for detection by a detector (e.g., a gamma detecting probe), an EGFR affinity moiety labeled with a detectable label (e.g., anti-EGFR antibodies of this invention labeled with a radioisotope, e.g., ¹⁶¹Tb, ¹²³I, ¹²⁵I, and the like), and, after allowing the active substance to be taken up by the target tissue, and preferably after blood clearance of the label, subjecting the mammal to a radioimmunodetection technique in the relevant area of the body, e.g., by using a gamma detecting probe.

In certain embodiments the chimeric moiety comprising an label-bound to an affinity moiety (e.g., C10 mutant antibody) of this invention can be used in the technique of radioguided surgery, wherein relevant tissues in the body of a subject can be detected and located intraoperatively by means of a detector, e.g., a gamma detecting probe. The surgeon can, intraoperatively, use this probe to find the tissues in which uptake of the compound labeled with a radioisotope, that is, e.g., a low-energy gamma photon emitter, has taken place.

In certain embodiments various preferred radiolabels include, but are not limited to ⁹⁹Tc, ²⁰³Pb, ⁶⁷Ga, ⁶⁸Ga, ⁷²As, ¹¹¹In, ^{113m}In, ⁹⁷Ru, ⁶²Cu, 641Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag.

Means of detecting such labels are well known to those of skill in the art. Thus, for example, radiolabels may be detected using photographic film, scintillation detectors, and the like. Fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

In certain specific embodiments the affinity moieties of this invention (e.g., C10 mutants) can be conjugated to gamma-emitting radioisotopes (e.g., Na-22, Cr-51, Co-60, Tc-99, I-125, I-131, Cs-137, GA-67, Mo-99) for detection with a gamma camera, to positron emitting isotopes (e.g., C-11, N-13, O-15, F-18, and the like) for detection on a Positron Emission Tomography (PET) instrument, and to metal contrast agents (e.g., Gd containing reagents, Eu containing reagents, and the like) for magnetic resonance imaging (MRI), In addition, the antibodies of this invention can be used in traditional immunohistochemistry (e.g., fluorescent labels, nanocrystal labels, enzymatic and colormetric labels etc.).

2) Radiosensitizers.

In another embodiment, the active agent can be a radiosensitizer that enhances the cytotoxic effect of ionizing radiation (e.g., such as might be produced by ⁶⁰Co or an x-ray source) on a cell. Numerous radiosensitizing agents are known and include, but are not limited to benzoporphyrin derivative compounds (see, e.g., U.S. Pat. No. 5,945,439), 1,2,4-benzotriazine oxides (see, e.g., U.S. Pat. No. 5,849,738), compounds containing certain diamines (see, e.g., U.S. Pat. No. 5,700,825), BCNT (see, e.g., U.S. Pat. No. 5,872,107), radiosensitizing nitrobenzoic acid amide derivatives (see, e.g., U.S. Pat. No. 4,474,814), various heterocyclic derivatives (see, e.g., U.S. Pat. No. 5,064,849), platinum complexes (see, e.g., U.S. Pat. No. 4,921,963), and the like.

3) Ligands.

In certain embodiments the active agent can also comprise a ligand, an epitope tag, or an antibody. Particularly preferred ligands and antibodies are those that bind to surface markers on immune cells. Chimeric molecules utilizing such antibodies as effector molecules act as bifunctional linkers establishing an association between the immune cells bearing binding partner for the ligand or antibody and the tumor cells expressing the EGFR family member(s).

In certain embodiments the active agent is an antibody that binds another epitope on EGFR or another member of the EGFR family. Attachment of the C10 mutant antibodies of this invention to such a second antibody produces a bispecific antibody. Suitable antibodies for such active agents include, but are not limited to C6.5, C6ML3-9, C6 MH3-B1, C6-B1D2, F5, HER3.A5, HER3.F4, HER3.H1, HER3.H3, HER3.E12, HER3.B12, EGFR.E12, EGFR.C10, EGFR.B11, EGFR.E8, HER4.B4, HER4.G4, HER4.F4, HER4.A8, HER4.B6, HER4.D4, HER4.D7, HER4.D11, HER4.D12, HER4.E3, HER4.E7, HER4.F8 and HER4.C7 (see, e.g., U.S. Pat. No. 7,332,585 and the PCT application WO07084181A2 both of which are incorporated herein by reference.

3) Chelates

Many pharmaceuticals and/or radiolabels described are provided as a chelate, particularly where a pre-targeting strategy is utilized. In such embodiments the chelating molecule is typically coupled to a molecule (e.g., biotin, avidin, streptavidin, etc.) that specifically binds an epitope tag attached to the EGFR affinity moiety (e.g., C10 mutant antibody).

Chelating groups are well known to those of skill in the art. In certain embodiments, chelating groups are derived from ethylene diamine tetra-acetic acid (EDTA), diethylene triamine penta-acetic acid (DTPA), cyclohexyl 1,2-diamine tetra-acetic acid (CDTA), ethyleneglycol-O,O′-bis(2-aminoethyl)-N,N,N′,N′-tetra-acetic acid (EGTA), N,N-bis(hydroxybenzyl)-ethylenediamine-N,N′-diacetic acid (HBED), triethylene tetramine hexa-acetic acid (TTHA), 1,4,7,10-tetraazacyclododecane-N,N′-,N″,N′″-tetra-acetic acid (DOTA), hydroxyethyldiamine triacetic acid (HEDTA), 1,4,8,11-tetra-azacyclotetradecane-N,N′,N″,N′″-tetra-acetic acid (TETA), substituted DTPA, substituted EDTA, and the like.

Examples of certain preferred chelators include unsubstituted or, substituted 2-iminothiolanes and 2-iminothiacyclohexanes, in particular 2-imino-4-mercaptomethylthiolane, and SAPS(N-(4-[211At] astatophenethyl) succinimate).

One chelating agent, 1,4,7,10-tetraazacyclododecane-N,N,N″,N′″-tetraacetic acid (DOTA), is of particular interest because of its ability to chelate a number of diagnostically and therapeutically important metals, such as radionuclides and radiolabels.

Conjugates of DOTA and proteins such as antibodies have been described. For example, U.S. Pat. No. 5,428,156 teaches a method for conjugating DOTA to antibodies and antibody fragments. To make these conjugates, one carboxylic acid group of DOTA is converted to an active ester which can react with an amine or sulfhydryl group on the antibody or antibody fragment. Lewis et al. (1994) Bioconjugate Chem. 5: 565-576, describes a similar method wherein one carboxyl group of DOTA is converted to an active ester, and the activated DOTA is mixed with an antibody, linking the antibody to DOTA via the epsilon-amino group of a lysine residue of the antibody, thereby converting one carboxyl group of DOTA to an amide moiety.

Alternatively the chelating agent can be coupled, directly or through a linker, to an epitope tag or to a moiety that binds an epitope tag. Conjugates of DOTA and biotin have been described (see, e.g., Su (1995) J. Nucl. Med., 36 (5 Suppl):154P, which discloses the linkage of DOTA to biotin via available amino side chain biotin derivatives such as DOTA-LC-biotin or DOTA-benzyl-4-(6-amino-caproamide)-biotin). Yau et al., WO 95/15335, disclose a method of producing nitro-benzyl-DOTA compounds that can be conjugated to biotin. The method comprises a cyclization reaction via transient projection of a hydroxy group; tosylation of an amine; deprotection of the transiently protected hydroxy group; tosylation of the deprotected hydroxy group; and intramolecular tosylate cyclization. Wu et al. (1992) Nucl. Med. Biol., 19(2): 239-244 discloses a synthesis of macrocylic chelating agents for radiolabeling proteins with ¹¹¹IN and ⁹⁰Y. Wu et al. makes a labeled DOTA-biotin conjugate to study the stability and biodistribution of conjugates with avidin, a model protein for studies. This conjugate was made using a biotin hydrazide which contained a free amino group to react with an in situ generated activated DOTA derivative.

4) Cytotoxins.

In various embodiments the active agent comprises a cytotoxin. Illustrative cytotoxins include, but are not limited to Pseudomonas exotoxins, Diphtheria toxins, ricin, abrin, and variants thereof.

Pseudomonas exotoxin A (PE) is an extremely active monomeric protein (molecular weight 66 kD), secreted by Pseudomonas aeruginosa, which inhibits protein synthesis in eukaryotic cells through the inactivation of elongation factor 2 (EF-2) by catalyzing its ADP-ribosylation (catalyzing the transfer of the ADP ribosyl moiety of oxidized NAD onto EF-2).

The toxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2, which inactivates the protein and causes cell death. The function of domain Ib (amino acids 365-399) remains undefined, although a large part of it, amino acids 365-380, can be deleted without loss of cytotoxicity. See Siegall et al. (1989) J. Biol. Chem. 264: 14256-14261.

Where the targeting molecule (e.g., C10 mutant antibody) is fused to PE, a preferred PE molecule is one in which domain Ia (amino acids 1 through 252) is deleted and amino acids 365 to 380 have been deleted from domain Ib. However all of domain Ib and a portion of domain II (amino acids 350 to 394) can be deleted, particularly if the deleted sequences are replaced with a linking peptide.

In addition, the PE molecules can be further modified using site-directed mutagenesis or other techniques known in the art, to alter the molecule for a particular desired application. Means to alter the PE molecule in a manner that does not substantially affect the functional advantages provided by the PE molecules described here can also be used and such resulting molecules are intended to be covered herein.

For maximum cytotoxic properties several modifications to the molecule can be made. An appropriate carboxyl terminal sequence to the recombinant molecule is preferred to translocate the molecule into the cytosol of target cells. Amino acid sequences which have been found to be effective include, REDLK (SEQ ID NO:32) (as in native PE), REDL (SEQ ID NO:33), RDEL (SEQ ID NO:34), or KDEL (SEQ ID NO:35), repeats of those, or other sequences that function to maintain or recycle proteins into the endoplasmic reticulum, referred to here as “endoplasmic retention sequences”. See, for example, Chaudhary et al. (1991) Proc. Natl. Acad. Sci. USA 87:308-312 and Seetharam et al, J. Biol. Chem. 266: 17376-17381. Preferred forms of PE comprise the PE molecule designated PE38QQR. (Debinski et al. Bioconj. Chem., 5: 40 (1994)), and PE4E (see, e.g., Chaudhary et al. (1995) J. Biol. Chem., 265: 16306).

Methods of cloning genes encoding PE fused to various ligands are well known to those of skill in the art (see, e.g., Siegall et al. (1989) FASEB J., 3: 2647-2652; and Chaudhary et al. (1987) Proc. Nat. Acad. Sci. USA, 84: 4538-4542).

Like PE, diphtheria toxin (DT) kills cells by ADP-ribosylating elongation factor 2 thereby inhibiting protein synthesis. Diphtheria toxin, however, is divided into two chains, A and B, linked by a disulfide bridge. In contrast to PE, chain B of DT, which is on the carboxyl end, is responsible for receptor binding and chain A, which is present on the amino end, contains the enzymatic activity (Uchida et al. (1972) Science, 175: 901-903; Uchida et al. (1973) J. Biol. Chem., 248: 3838-3844).

In a preferred embodiment, the targeting molecule-Diphtheria toxin fusion proteins of this invention have the native receptor-binding domain removed by truncation of the Diphtheria toxin B chain. Particularly preferred is DT388, a DT in which the carboxyl terminal sequence beginning at residue 389 is removed. Chaudhary et al. (1991) Bioch. Biophys. Res. Comm., 180: 545-551. Like the PE chimeric cytotoxins, the DT molecules may be chemically conjugated to the MUC-1 antibody, but, in certain preferred embodiments, the targeting molecule will be fused to the Diphtheria toxin by recombinant means (see, e.g., Williams et al. (1990) J. Biol. Chem. 265: 11885-11889).

5) Pharmaceuticals.

In certain embodiments the active agent comprises one or more pharmaceuticals. Suitable pharmaceuticals include, but are not limited to anti-cancer pharmaceuticals. One useful class of anti-cancer pharmaceutical includes the retinoids. Retinoids are useful in treating a wide variety of epithelial cell carcinomas, including, but not limited to pulmonary, head, neck, esophagus, adrenal, prostate, ovary, testes, pancreas, and gut.

Retinoic acid, analogues, derivatives, and mimetics are well known to those of skill in the art. Such retinoids include, but are not limited to retinoic acid, ceramide-generating retinoid such as fenretinide (see, e.g., U.S. Pat. No. 6,352,844), 13-cis retinoic acid (see, e.g., U.S. Pat. Nos. 6,794,416, 6,339,107, 6,177,579, 6,124,485, etc.), 9-cis retinoic acid (see, e.g., U.S. Pat. Nos. 5,932,622, 5,929,057, etc.), 9-cis retinoic acid esters and amides (see, e.g., U.S. Pat. No. 5,837,728), 11-cis retinoic acid (see, e.g., U.S. Pat. No. 5,719,195), all trans retinoic acid (see, e.g., U.S. Pat. Nos. 4,885,311, 4,994,491, 5,124,356, etc.), 9-(Z)-retinoic acid (see, e.g., U.S. Pat. Nos. 5,504,230, 5,424,465, etc.), retinoic acid mimetic anlides (see, e.g., U.S. Pat. No. 6,319,939), ethynylheteroaromatic-acids having retinoic acid-like activity (see, e.g., U.S. Pat. Nos. 4,980,484, 4,927,947, 4,923,884 Ethynylheteroaromatic-acids having retinoic acid-like activity, U.S. Pat. No. 4,739,098, etc.) aromatic retinoic acid analogues (see, e.g., U.S. Pat. No. 4,532,343), N-heterocyclic retinoic acid analogues (see, e.g., U.S. Pat. No. 4,526,7874), naphtenic and heterocyclic retinoic acid analogues (see, e.g., U.S. Pat. No. 518,609), open chain analogues of retinoic acid (see, e.g., U.S. Pat. No. 4,490,414), entaerythritol and monobenzal acetals of retinoic acid esters (see, e.g., U.S. Pat. No. 4,464,389), naphthenic and heterocyclic retinoic acid analogues (see, e.g., U.S. Pat. No. 4,456,618), azetidinone derivatives of retinoic acid (see, e.g., U.S. Pat. No. 4,456,618), and the like.

In various embodiments the retinoic acid, retinoic acid analogue, derivative, or mimetics can be coupled (e.g., conjugated) to the targeting component (e.g. C10 mutant antibody) or it can be contained within a liposome or complexed with a lipid or a polymeric nanoparticle that is coupled to the targeting moiety, e.g. as described herein.

In certain embodiments the methods and compositions of this invention can be used to deliver other cancer therapeutics instead of or in addition to the retinoic acid or retinoic acid analogue/derivative. Such agents include, but are not limited to alkylating agents (e.g., mechlorethamine (Mustargen), cyclophosphamide (Cytoxan, Neosar), ifosfamide (Ifex), phenylalanine mustard; melphalen (Alkeran), chlorambucol (Leukeran), uracil mustard, estramustine (Emcyt), thiotepa (Thioplex), busulfan (Myerlan), lomustine (CeeNU), carmustine (BiCNU, BCNU), streptozocin (Zanosar), dacarbazine (DTIC-Dome), cis-platinum, cisplatin (Platinol, Platinol AQ), carboplatin (Paraplatin), altretamine (Hexylen), etc.), antimetabolites (e.g. methotrexate (Amethopterin, Folex, Mexate, Rheumatrex), 5-fluoruracil (Adrucil, Efudex, Fluoroplex), floxuridine, 5-fluorodeoxyuridine (FUDR), capecitabine (Xeloda), fludarabine: (Fludara), cytosine arabinoside (Cytaribine, Cytosar, ARA-C), 6-mercaptopurine (Purinethol), 6-thioguanine (Thioguanine), gemcitabine (Gemzar), cladribine (Leustatin), deoxycoformycin; pentostatin (Nipent), etc.), antibiotics (e.g. doxorubicin (Adriamycin, Rubex, Doxil, Daunoxome-liposomal preparation), daunorubicin (Daunomycin, Cerubidine), idarubicin (Idamycin), valrubicin (Valstar), mitoxantrone (Novantrone), dactinomycin (Actinomycin D, Cosmegen), mithramycin, plicamycin (Mithracin), mitomycin C (Mutamycin), bleomycin (Blenoxane), procarbazine (Matulane), etc.), mitotic inhibitors (e.g. paclitaxel (Taxol), docetaxel (Taxotere), vinblatine sulfate (Velban, Velsar, VLB), vincristine sulfate (Oncovin, Vincasar PFS, Vincrex), vinorelbine sulfate (Navelbine), etc.), chromatin function inhibitors (e.g., topotecan (Camptosar), irinotecan (Hycamtin), etoposide (VP-16, VePesid, Toposar), teniposide (VM-26, Vumon), etc.), hormones and hormone inhibitors (e.g. diethylstilbesterol (Stilbesterol, Stilphostrol), estradiol, estrogen, esterified estrogens (Estratab, Menest), estramustine (Emcyt), tamoxifen (Nolvadex), toremifene (Fareston) anastrozole (Arimidex), letrozole (Femara), 17-OH-progesterone, medroxyprogesterone, megestrol acetate (Megace), goserelin (Zoladex), leuprolide (Leupron), testosteraone, methyltestosterone, fluoxmesterone (Android-F, Halotestin), flutamide (Eulexin), bicalutamide (Casodex), nilutamide (Nilandron), etc.) INHIBITORS OF SYNTHESIS (e.g., aminoglutethimide (Cytadren), ketoconazole (Nizoral), etc.), immunomodulators (e.g., rituximab (Rituxan), trastuzumab (Herceptin), denileukin diftitox (Ontak), levamisole (Ergamisol), bacillus Calmette-Guerin, BCG (TheraCys, TICE BCG), interferon alpha-2a, alpha 2b (Roferon-A, Intron A), interleukin-2, aldesleukin (ProLeukin), etc.) and other agents such as 1-aspariginase (Elspar, Kidrolase), pegaspasgase (Oncaspar), hydroxyurea (Hydrea, Doxia), leucovorin (Wellcovorin), mitotane (Lysodren), porfimer (Photofrin), tretinoin (Veasnoid), and the like.

6) Ribozymes

In certain embodiments the active agents include ribozymes (see, e.g., Scanlon (2004) Curr Pharm Biotechnol., 5: 415-420; Citti and Rainaldi (2005) Curr Gene Ther., 5: 11-24.). The ribozymes are typically provided encapsulated in a liposome or nanocapsule or admixed in a lipid. In addition to possessing catalytic activities as well as binding capacity to the RNA, the hammerhead ribozymes can cause RNase-dependent degradation of the target double-stranded RNA (dsRNA). Ribozymes can be directed against a number of different targets in the treatment of a cancer. Thus for example, a modified chimeric ribozyme targeting VEGF receptor, flt-1 (Angiozyme), was developed by Ribozyme Inc., which is now renamed Sima Therapeutics Inc. (Boulder, Colo.).

7) Antisense/Antigene Molecules.

In certain embodiments the active agents include antisense and/or antigene molecules. Antigene oligonucleotides are antisense sequences that can insert themselves into a section of a DNA to form a triple helix, and thus inhibit transcription. Recognition of a duplex sequence by a third strand of DNA or RNA via the major groove is the basis of the formation of a triple helix. Typically, stable triplexes form on polypurine:polypyrimidine tracts. The third strand, depending on the target sequence, may consist of purines or pyrimidines, and the complex is stabilized by two Hoogsteen hydrogen bonds between third strand bases and the bases in the purine strand of the duplex. Triple helix is an inherent property of DNA and requires no additional enzymes or proteins.

Peptide nucleic acids (PNAs) are DNA analogs consisting of nucleobases attached to a peptide backbone of N-(2-aminoethyl)glycine residues. The phosphate charges are replaced with neutral peptide linkage, resulting in a stable hybrid between PNA and DNA or RNA strands. In addition, they can form triplexes by Hoogsteen pairing on polypurine and polypyrimidine targets. PNAs are resistant to degradation, form stable complexes on DNA targets and show high sequence selectivity, making them very attractive for cancer therapy (see, e.g., Dean (2000) Adv Drug Deliv Rev, 44: 81-95; Nielsen (2001) Curr Med Chem 8: 545-550; Braasch and Corey (2002) Biochemistry 41: 4503-4510; and the like.).

Antisense oligonucleotides are the most widely used unmodified or chemically modified single-stranded RNA or DNA molecules. One of the first reports to show in vivo activity was of a phosphodiester oligonucleotide directed against N-MYC that caused a decrease in tumor mass associated with loss of N-MYC protein in a subcutaneously transplanted neuroepithelioma in mice (Whitesell et al. (1991) Antisense Res Dev 1: 343-350). As the phosphodiester bond is highly susceptible to degradation, the development of phosphorothioate chemistry, which contains a sulfur atom in each internucleotide linkage instead of oxygen, revolutionized this field because of its stability (Lebedeva et al. (2001) Annu Rev Pharmacol Toxicol 41: 403-419; Crooke (2004) Annu Rev Med 55: 61-95; and the like).

The phosphorothioate antisense has shown the broadest range of activity in preclinical and clinical studies (ISIS Pharmaceuticals Inc., Carlsbad, Calif.; Genta Inc., Berkeley Heights, N.J.; Hybridon Inc., Cambridge, Mass.).

Certain second-generation antisense oligonucleotides comprise alkyl modifications at the 2′ position of the ribose and the development of novel chemically modified nucleotides with improved properties such as enhanced serum stability, higher target affinity and low toxicity (Kurreck (2003) Eur J Biochem 270: 1628-1644). One such modification in oligomer chemistry has led to the development of the phosphorodiamidate morpholino oligomers (PMO) by AVI BioPharma Inc. (Portland, Oreg.), which are non-ionic antisense agents that inhibit gene expression by binding to RNA and sterically blocking processing or translation in an RNaseH-independent manner. PMO antisense agents have revealed excellent safety profile and efficacy in multiple disease models including cancer preclinical studies targeting for example, c-myc, and/or MMP-9 (see, e.g., Hudziak et al. (2000) Antisense Nucleic Acid Drug Dev 10: 163-176; Devi et al. (2002) Prostate 53: 200-210; Knapp et al. (2003) Anticancer Drugs 14: 39-47; London et al. (2003) Cancer Gene Ther 10: 823-832; Devi (2002) Curr Opin Mol Ther 4: 138-148; Ko et al. (2004) J. Urol. 172: 1140-1144; Iversen et al. (2003) Clin Cancer Res 9: 2510-2519; and the like).

8) RNAi.

In certain embodiments the nanoparticles of this invention can be used to deliver an siRNA. Preclinical cancer studies have shown inhibition of growth and survival of tumor cells by RNAi-mediated downregulation of several key oncogenes or tumor-promoting genes, including growth and angiogenic factors or their receptors (vascular endothelial growth factor, epidermal growth factor receptor), human telomerase (hTR, hTERT), viral oncogenes (papillomavirus E6 and E7) or translocated oncogenes (BCR-abl). Various studies are reporting in vivo activity and the potential of RNAi to suppress tumor growth. These include an intratumoral injection of an shRNA-adenoviral vector construct targeting a cell cycle regulator causing inhibition of subcutaneous small cell lung tumor in mice, and systemic administration of an siRNA targeting a carcinoembryonic antigen-related cell adhesion molecule (CEACAM6) in mice with subcutaneously xenografted pancreatic adenocarcinoma cells. In another report, direct injection of a plasmid vector expressing shRNAs to matrix metalloproteinase MMP-9 and a cathepsin showed efficacy in established glioblastoma.

Illustrative targets for siRNA as a cancer therapeutic include, but are not limited to Bax or Bcl-2 targeting the apoptosis pathway (see, e.g., Grzmil et al. (2003) Am J. Pathol., 163: 543-552; Yin et al. (2003) J Exp Ther Oncol., 3: 194-204), focal adhesion kinase (FAK targeting angiogenesis) (see, e.g., Duxbury (2003) Biochem Biophys Res Commun., 311: 786-792) adhesion matrix metalloproteinase (Sanceau (2003) J Biol Chem 278: 36537-36546), VEGF (see, e.g., Yin et al. (2003) J Exp Ther Oncol. 3:194-204; Zhang (2003) Biochem Biophys Res Commun., 303: 1169-1178), fatty acid synthase (De Schrijver et al. (2003) Cancer Res., 63: 3799-3804.), MDR (Nieth et al. (2003) FEBS Lett., 545: 144-150), H-Ras (Yin et al. (2003) J Exp Ther Oncol. 3: 194-204; Zhang (2003) Biochem Biophys Res Commun., 303:1169-1178), K-Ras (Lois et al (2001) Curr Opin Immunol., 13: 496-504), PLK-1 (Spankuch-Schmitt et al. (2002) J Natl Cancer Inst., 94: 1863-1877), TGF-β (Yin et al. (2003) J Exp Ther Oncol. 3:194-204) STAT3 (Konnikova et al. (2003) BMC Cancer 3: 23) EGFR (Nagy et al. (2003) Exp Cell Res., 285: 39-49; Zhang et al. (2004) Acta Pharmacol., 25: 61-67), PKC-α (Yin et al. (2003) J Exp Ther Oncol. 3: 194-204) Epstein-Barr virus (Li et al. (2004) Biochem Biophys Res Commun., 315: 212-218) HPV E6 (Butz et al. (2003) Oncogene 22: 5938-5945), BCR-Abl (Wohlbold et al. (2003) Blood 102: 2236-2239; Fuchs et al. (2002) Oncogene, 21: 5716-5724), telomerase (Kosciolek et al. (2003) Mol Cancer Ther. 2: 209-216), and the like.

V. Administration of Antibodies, and/or Chimeric Moieties.

A) Pharmaceutical Formulations.

In certain embodiments the antibodies, and/or affinity moiety/microparticle or nanoparticle constructs of the present invention can be formulated as pharmaceutical compositions (i.e., compositions that are suitable for administration to a subject or patient (i.e., human or non-human subject) that can be used directly and/or in the preparation of unit dosage forms. In certain embodiments, such compositions comprise a therapeutically effective amount of one or more therapeutic agents (e.g., C10 mutant antibody, microparticle, nanoparticle, etc.) and a pharmaceutically acceptable carrier.

As indicated above, the agents of this invention can be used in a wide variety of contexts including, but not limited to the detection and/or imaging of tumors or cancer cells, inhibition of tumor growth and/or cancer cell growth and/or proliferation, and the like. One or more antibodies, and/or chimeric moieties of this invention can be administered by injection, that is, intravenously, intramuscularly, intracutaneously, subcutaneously, intraduodenally, or intraperitoneally. Also, in certain embodiments, the compounds can be administered by inhalation, for example, intranasally. Additionally, certain compounds can be administered orally, or transdermally.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans, or suitable for administration to an animal or human. The term “carrier” or refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of the compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments the compositions of the invention can be provided as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Pharmaceutical compositions comprising the moieties described herein can be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the molecules into preparations that can be used pharmaceutically. Proper formulation is dependent upon the route of administration chosen.

For topical or transdermal administration, the moieties described herein can be formulated as solutions, gels, ointments, creams, lotion, emulsion, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal, inhalation, oral or pulmonary administration. In the context of treatment of neoplasms, intratumoral injections can be performed. One advantageous method for local administration of the described moieties is intracranial infusion by convection-enhanced delivery to the brain.

For injection, the moieties described herein can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, compositions comprising the iron chelating agent(s) can be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the antibodies, and/or chimeric moieties of this invention can be readily formulated by combining the agent(s) with pharmaceutically acceptable carriers well known in the art. Such carriers enable the agent(s) to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g., lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added.

For buccal administration, the iron chelating agent(s) can take the form of tablets, lozenges, etc. formulated in conventional manner.

For administration by inhalation, antibodies, and/or chimeric moieties of this invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator may be formulated containing a powder mix of the iron chelating agent(s) and a suitable powder base such as lactose or starch.

The antibodies, and/or chimeric moieties of this invention (can also be formulated in rectal or vaginal compositions such as suppositories or retention enemas, e.g, containing conventional suppository bases such as cocoa butter or other glycerides.

In addition to the formulations described previously, the antibodies, and/or chimeric moieties of this invention can also be formulated as a depot preparation. Such long acting formulations may be administered by implantation (for example subcutaneously or intramuscularly) or by intramuscular injection. Thus, for example, the agent(s) of this invention can be formulated with suitable polymeric or hydrophobic materials (for example as an emulsion in an acceptable oil) or ion exchange resins, or as sparingly soluble derivatives, for example, as a sparingly soluble salt.

Other pharmaceutical delivery systems can also be employed. Liposomes and emulsions are well known examples of delivery vehicles that may be used to deliver the antibodies, and/or chimeric moieties of this invention. Certain organic solvents such as dimethylsulfoxide also may be employed, although usually at the cost of greater toxicity. Additionally, the antibodies, and/or functionalized chimeric moieties of this invention can be delivered using a sustained-release system, such as semipermeable matrices of solid polymers containing the therapeutic agent. Various sustained-release materials have been established and are well known by those skilled in the art. Sustained-release capsules may, depending on their chemical nature, can release the active agent(s) for a few days, a few weeks, or up to over 100 days. Depending on the chemical nature and the biological stability of the agent(s) additional strategies for stabilization can be employed.

As the antibodies, and/or chimeric moieties of this invention may contain charged side chains or termini, they can be included in any of the above-described formulations as the free acids or bases or as pharmaceutically acceptable salts. Pharmaceutically acceptable salts are those salts which substantially retain the biological activity of the free bases and which are prepared by reaction with inorganic acids. Pharmaceutical salts tend to be more soluble in aqueous and other protic solvents than are the corresponding free base forms.

B) Effective Dosages.

The antibodies, antibodies, and/or chimeric moieties of this invention will generally be used in an amount effective to achieve the intended purpose (e.g., to image a tumor or cancer cell, to inhibit growth and/or proliferation of cancer cells, etc.). In certain preferred embodiments, the antibodies, and/or chimeric moieties utilized in the methods of this invention are administered at a dose that is effective to partially or fully inhibit cancer cell proliferation and/or growth, or to enable visualization of a cancer cell or tumor characterized by overexpression of an EGF receptor. In certain embodiments, dosages are selected that inhibit cancer cell growth and/or proliferation at the 90%, more preferably at the 95%, and most preferably at the 98% or 99% confidence level. Preferred effective amounts are those that reduce or prevent tumor growth or that facilitate cancer cell detection and/or visualization. With respect to inhibitors of cell growth and proliferation, the compounds can also be used prophalactically at the same dose levels.

Typically, the antibodies, and/or chimeric moieties of this invention, or pharmaceutical compositions thereof, are administered or applied in a therapeutically effective amount. A therapeutically effective amount is an amount effective to reduce or prevent the onset or progression (e.g., growth and/or proliferation) of a cancer cell and/or a tumor. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One skilled in the art could readily optimize administration to humans based on animal data.

Dosage amount and interval can be adjusted individually to provide plasma levels of the inhibitors which are sufficient to maintain therapeutic effect.

Dosages for typical therapeutics are known to those of skill in the art. Moreover, such dosages are typically advisorial in nature and may be adjusted depending on the particular therapeutic context, patient tolerance, etc. Single or multiple administrations of the compositions may be administered depending on the dosage and frequency as required and tolerated by the patient.

In certain embodiments, an initial dosage of about 1 μg, preferably from about 1 mg to about 1000 mg per kilogram daily will be effective. A daily dose range of about 5 to about 75 mg is preferred. The dosages, however, can be varied depending upon the requirements of the patient, the severity of the condition being treated, and the compound being employed. Determination of the proper dosage for a particular situation is within the skill of the art. Generally, treatment is initiated with smaller dosages that are less than the optimum dose of the compound. Thereafter, the dosage is increased by small increments until the optimum effect under the circumstance is reached. For convenience, the total daily dosage can be divided and administered in portions during the day if desired. Typical dosages will be from about 0.1 to about 500 mg/kg, and ideally about 25 to about 250 mg/kg.

In cases of local administration or selective uptake, the effective local concentration of the antibodies and/or chimeric moieties may not be related to plasma concentration. One skilled in the art will be able to optimize therapeutically effective local dosages without undue experimentation. The amount of antibody and/or chimeric moiety will, of course, be dependent on the subject being treated, on the subject's weight, the severity of the affliction, the manner of administration and the judgment of the prescribing physician.

The therapy can be repeated intermittently. In certain embodiments, the pharmaceutical preparation comprising the antibodies and/or chimeric moieties can be administered at appropriate intervals, for example, at least twice a day or more until the pathological symptoms are reduced or alleviated, after which the dosage may be reduced to a maintenance level. The appropriate interval in a particular case would normally depend on the condition of the patient. The therapy can be provided alone or in combination with other drugs, and/or radiotherapy, and/or surgical procedures.

C) Toxicity.

Preferably, a therapeutically effective dose of antibodies, and/or chimeric moieties of this invention described herein will provide therapeutic benefit without causing substantial toxicity.

Toxicity of the agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Agents that exhibit high therapeutic indices are preferred. Data obtained from cell culture assays and animal studies can be used in formulating a dosage range that is not toxic for use in human. The dosage of the antibodies, and/or chimeric moieties of this invention preferably lie within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition (see, e.g., Fingl et al. (1975) In: The Pharmacological Basis of Therapeutics, Ch. 1, p. 1).

### VI. Kits.

The present invention further encompasses kits for use in detecting cells expressing or overexpressing the EGF receptor in vivo, and/or in biological samples. Kits are also provided for in inhibiting the growth and/or proliferation of cells expressing or overexpressing EGFR (e.g., cancer cells).

In certain embodiments, the kits comprise one or more antibodies, and/or chimeric moieties of this invention. In certain preferred embodiments, the antibodies are scFv antibodies. Depending on use, the antibodies can be functionalized with linkers and/or chelators for coupling to an effector (e.g., a radioactive moiety, a liposome, a cytotoxin, another antibody, etc.) as described herein.

In certain embodiments, the kits can comprise the molecules of the invention specific for EGFR as well as buffers and other compositions to be used for detection of the molecules.

The kits can also include instructional materials teaching the use of the antibodies for detecting, e.g., cancer cells, and/or teaching the combination of the antibodies with functionalizing reagents or teaching the use of functionalized antibodies for imaging and/or therapeutic applications. In certain embodiments, the antibody is provided functionalized with a linker and/or a chelator (in one container) along with one or more effectors, e.g., cytotoxins, radioactive labels (in a second container) such that the two components can be separately administered (e.g., in pre-targeting approaches) or such that the two components can be administered shortly before use.

Certain instructional materials will provide recommended dosage regimen, counter indications, and the like. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such.

### EXAMPLES

The following examples are offered to illustrate, but not to limit the claimed invention.

### Example 1

### Impact of Single-Chain FV Antibody Fragment Affinity on Nanoparticle Targeting of Epidermal Growth Factor Receptor-Expressing Tumor Cells

To determine the importance of single-chain Fv (scFv) affinity on binding, uptake, and cytotoxicity of tumor-targeting nanoparticles, the affinity of the epidermal growth factor receptor (EGFR) scFv antibody C10 was increased using molecular evolution and yeast display. A library containing scFv mutants was created by error-prone PCR, displayed on the surface of yeast, and higher affinity clones selected by fluorescence activated cell sorting. Ten mutant scFv were identified that had a 3-18-fold improvement in affinity (KD=15-88 nM) for EGFR-expressing A431 tumor cells compared to C10 scFv (KD=264 nM). By combining mutations, higher affinity scFv were generated with KD ranging from 0.9 nM to 10 nM. The highest affinity scFv had a 280-fold higher affinity compared to that of the parental C10 scFv. Immunoliposome nanoparticles (ILs) were prepared using EGFR scFv with a 280-fold range of affinities, and their binding and uptake into EGFR-expressing tumor cells was quantified. At scFv densities greater than 148 scFv/IL, there was no effect of scFv affinity on IL binding and uptake into tumor cells, or on cytotoxicity. At lower scFv densities, there was less uptake and binding for ILs constructed from the very low affinity C10 scFv. The results show the importance of antibody fragment density on nanoparticle uptake, and suggest that engineering ultrahigh affinity scFv may be unnecessary for optimal nanoparticle targeting.

### Materials and Methods

Cell Lines, Media, Antibodies and Recombinant EGFR-ECD

Yeast strain EBY100 (GAL1-AGA1::URA3 ura3-52 trp1 leu2Δ1 his3 A200 pep4::HIS2 prb1Δ1.6R can1 GAL) was grown in YPD medium, and EBY100 transfected with expression vector pYD226 was selected on SD-CAA medium. The Aga2p scFv fusion was expressed on the yeast surface by induction in SG-CAA medium (SD-CAA medium with glucose replaced by galactose) at 20° C. for 24˜48 h as described (Feldhaus et al. (2003) Nature Biotechnol. 21: 163-170). Bacteria strains E. coli DH5α (K12, ΔlacU169 (Φ80 lacZΔM15), supE44, hsdR17, recA1, endA1, gyrA96, thi-1, relA1) and TG1 (K12, Δ(lac-pro), supE, thi, hsdD5/F′ traD36, proA+B+, lacIq, lacZΔM15) were used for the preparation of plasmid DNA and the expression of soluble scFv antibodies, respectively. The A431 epidermal cancer cell line, MDAMB468, MDAMB453 and MDAMB231 breast carcinoma cell lines were obtained from the University of California San Francisco Cell Culture Facility. U87 and U87vIII human glioblastoma cancer cell lines were obtained from the American Type Culture Collection. NR6 and stable EGFR (vIII)-transfected NR6M cells were kindly provided by Dr Daryl D Bigner.53 A431 cells were maintained in RPMI 1640 medium, while MDAMB231, U87, U87vIII, NR6, and NR6M cells were grown in DME-H21 medium supplemented with 10% (v/v) fetal bovine serum, in a humidified atmosphere of 95% air, 5% CO2 at 37° C. MDAMB468 and MDAMB453 cells were grown in Leiboviz′ 15 (Li5) medium with 10% fetal bovine serum, in humidified air at 37° C. SV5 antibody was purified from the hybridoma supernatant using protein G, and labeled directly with Alexa-488 using a kit provided by the manufacturer (Invitrogen; Carlsbad, Calif.). Recombinant EGFR-ECD was expressed in HEK293 cells (Horak et al. (2005) Cancer Biother. Radiopharm. 20: 603-613). The functional EGFR-ECD was determined to be 10% active with respect to antibody binding by BIAcore. EGFR-ECD was biotinylated with NHS-sulfo-LC-biotin following the protocol provided by the manufacturer (Pierce; Rockford, Ill.).

Materials for Liposome Preparation

DiIC¹⁸(3)-DS was purchased from Molecular Probes (Eugene, Oreg.). Distearoylphosphatidylcholine (DSPC) and poly(ethylene)glycol (PEG2000)-derivatized distearoylphosphatidylethanolamine (PEG₂₀₀₀-DSPE) were purchased from Avanti Polar Lipids (Alabaster, Ala.). Cholesterol was obtained from Calbiochem (La Jolla, Calif.). Topotecan was a gift from the Taiwan Liposome Company (Taipei, Taiwan) and doxorubicin hydrochloride (Bedford Laboratories; Bedford, Ohio) was obtained from the pharmacy. Sucrose octasulfate (sodium salt) was purchased from Toronto Research Chemicals, Inc. (North York, ON, Canada). Sepharose CL-4B and Sephadex G-75 size-exclusion resins, Dowex 50W-8X-200 cation-exchange resin, and triethylamine were all obtained from Sigma-Aldrich (St. Louis, Mo.).

Construction, Expression and Characterization of scFv Mutant Yeast Display Library

Random mutations were introduced into the anti-EGFR scFv C10 gene by error-prone PCR as described (Daugherty et al. (2000) Proc. Natl. Acad. Sci. USA, 97: 2029-2034). Briefly, the anti-EGFR scFv C10 gene in the pYD2 expression vector was subjected to 20 cycles of error-prone PCR using primers:

| | |
|---|---|
| pYD1F | |
| (5′-AGTAACGTTTGTCAGTAATTGC-3′) | (SEQ ID NO: 36) |
| pYD1R | |
| (5′-GTCGATTTTGTTACATCTACAC-3′) | (SEQ ID NO: 37) |

in a reaction mixture with 0.5 mM MnCl₂. The mutated scFv gene was re-amplified by non-error-prone PCR with primers:

| | |
|---|---|
| Gap5 (5′-TTAAGCTTCTGCAGGCTAGTG-3′) | (SEQ ID NO: 38) |
| Gap3 (5′-GAGACCGAGGAGAGGGTTAGG-3′) | (SEQ ID NO: 39) |

and a high-fidelity DNA polymerase. Approximately 1 μg of the amplified gene was precipitated with ethanol and used to transform lithium acetate-treated EBY100 cells together with 2 μg of NcoI/NotI-digested vector pYD2 using the TRAFO method with Gap Repair (Gietz and Schiestl (1991) Yeast, 7: 253-263; Orr-Weaver and Szostak (1983) Proc. Natl. Acad. Sci. USA, 80: 4417-4421). Transformation mixes were cultured and subcultured in SD-CAA medium. The library size was calculated by plating serial dilutions of the transformed cells on SD-CAA plates. The error rate was estimated by sequencing the entire scFv gene isolated from randomly picked colonies.

Cell Labeling and Sorting with Mutant scFv Library

The transformed culture was induced in SG-CAA medium for 24 h at 20° C. For sorting, the amount of yeast stained was always at least ten times greater than the library size or the maximum diversity present based on previous sort rounds. For staining, yeast were washed and resuspended in fluorescent-activated cell sorting (FACS) buffer (phosphate-buffered saline (PBS) (pH 7.4), 0.5% (w/v) bovine serum albumin) containing the desired concentration of biotinylated EGFR-ECD. Incubation time and volume were set to ensure the reaction had reached equilibrium (Razai et al. (2005) J. Mol. Biol. 351: 158-169). After incubation, cells were washed three times with ice-cold FACS buffer and resuspended in a 1:400 (v/v) dilution of 1 mg/ml SV5-488, and either a 1:800 (v/v) dilution of streptavidin PE (Biosource) or neutravidin PE (Molecular Probes). Before sorting with a FACSAria instrument, the stained cells were kept on ice for 30 min, pelleted by centrifugation (GH-3.8A rotor, 200 g, 5 min, 4° C.), and resuspended in FACS buffer at 1×10⁶-5×10⁶ cells/ml. The displayed C10 mutant library was subjected to four rounds of selection, with the first two rounds in yield mode, followed by two rounds of selection in the purity mode. The sort gate was set to recover 0.5% of the labeled cells. Collected cells were plated on SD-CAA plates, recovered by scraping colonies from the plate, cultured in SD-CAA and used for the next round of sorting after induction in SG-CAA. Concentrations of biotinylated EGFR-ECD used for sorting were 8 μM, 1 μM, 250 nM, and 100 nM, for the first through fourth rounds, respectively.

Site-Directed Mutagenesis

Primers G→ARev, 5′-AGCCGCATAGCAGCTGGTACT-3′(SEQ ID NO:40) G→A For, 5′-ACCAGCTGCTATGCGGCTTTTGATATCTGG-3 (SEQ ID NO:41) were used to introduce the site-specific mutation Glycine to alanine in the heavy chain CDR3 into the scFv genes. Briefly, the scFv gene was used as a template for PCR amplification with primers Gap5 and G→ARev for the heavy chain fragment and primers G→A For and Gap3 for the light chain fragment. Both fragments at concentration of 6 μg/μl were spliced together in a 25 μl PCR reaction using a high-fidelity DNA polymerase for ten cycles. The spliced scFv gene was used to transform EBY100. Individual yeast colonies were characterized for the G→A mutation by DNA sequencing.

Measurement of Yeast-Displayed scFv Affinity for Biotinylated EGFR-ECD

Quantitative equilibrium binding was determined using yeast-displayed scFv and flow cytometry as described (Boder et al. (2000) Proc. Natl. Acad. Sci. USA, 97: 10701-10705). Generally, six to eight different concentrations of biotinylated EGFR were used to span a range of concentrations from ten times above to ten times below the K_{D}. Incubation volumes, times and yeast numbers were chosen to ensure that the studies were done in at least fivefold antigen excess and that equilibrium had been achieved (Razai et al. (2005) J. Mol. Biol. 351: 158-169). For anti-EGFR scFv, 105 yeast in 50 μl were incubated with biotinylated EGFR-ECD for 1 h at room temperature. Binding of biotinylated EGFR-ECD to yeast-displayed scFv was detected using a 1:800 (v/v) dilution of streptavidin PE. Only yeast displaying scFv (as determined by binding of SV5 mAb) were gated for affinity measurements.

Expression and Purification of Soluble scFv from Yeast Displayed scFv

To generate soluble scFv antibodies, the scFv genes were subcloned from the pYD2 vector into the pSYN1 vector for expression in bacteria TG1 (Schier et al. (1995) Immunotechnology, 1: 73-81). The plasmids of pYD2-scFv were extracted from yeast and transformed into bacteria DH5α. Plasmid DNA was prepared from DH5α, digested with NcoI and NotI, and the scFv gene was gel-purified and ligated into NcoI/NotI-digested pSYN1 vector. E. coli TG1 cells were transformed with the pSYN1-scFv ligation mixture. Transformed TG1 cells were cultured and scFv expression induced by adding 0.1 mM IPTG as described (Id.). scFv antibodies were purified from the osmotic shock fractions by using a Ni-NTA agarose column (Id.). The monomeric scFv fractions were isolated from dimeric and aggregated scFv by gel filtration chromatography using a Sephadex G-75 column (Id.). scFv constructs with a free cysteine residue at the COOH terminus for conjugation to liposomes were created, expressed, and purified as described (Liu et al. (2004) Cancer Res. 64: 704-710).

Measurement of scFv Affinity for Cells Expressing EGFR

Human squamous carcinoma A431 cells expressing EGFR were grown to 80-90% confluence in RPMI medium supplemented with 10% fetal bovine serum and harvested by trypsinization. Each scFv was incubated overnight with 5×10⁴ cells at a range of concentrations from tenfold above to tenfold below the KD. Cell binding was performed at 4° C. in FACS buffer in a volume calculated to ensure fivefold scFv excess and for a duration calculated to ensure that the reaction had reached equilibrium. After two washes with 200 μL of FACS buffer, cell-bound scFv was detected by the addition of 100 μl of 1 μg/ml biotinylated His probe (Santa Cruz Biotech.) and a 1:800 (v/v) dilution of streptavidin-PE (Biosource). After incubation at 4° C. for 30 min, the cells were washed twice and resuspended in PBS containing 4% (v/v) paraformaldehyde. Fluorescence was measured by flow cytometry in a FACS LSRII instrument (Becton Dickinson), and the median fluorescence intensity (MFI) values were fit to the equation:

MFI=MFIₘᵢₙ+MFIₘₐₓ([Ab]/(K_{D}+[Ab])

using the software program Kaleidagraph as described previously (Benedict et al. (1997) J. Immunol. Methods, 201: 223-231).

Preparation of Fluorescent and Drug-Loaded Liposomes

Fluorescence-labeled unilamellar liposomes were prepared by the repeated freeze-thawing method (Szoka and Papahadjopoulos (1980) Annu. Rev. Biophys. Bioeng. 9: 467-508), using DSPC and cholesterol (molar ratio 3:2) with N-(polyethylene glycol)distearoylphosphatidylethanolamine (PEG-DSPE) (0.5-5 mol % of phospholipid). Liposomes were subsequently extruded 10-15 times through polycarbonate filters with 0.1 μm pore size, yielding liposomes of 100-120 nm diameter as determined by dynamic light-scattering. Concentrations of liposomal phospholipid (PL) were determined using a standard phosphate assay (Bartlett (1959) J. Biol. Chem. 234: 466-468). For uptake and internalization studies, liposomes were labeled with 0.5 mol % DiIC18(3)-DS [1,1-Dioctadecyl-3,3,3,3-tetramethylindocarbocyanine-5,5-disulfonic acid] (Invitrogen/Molecular Probe), a fluorescent lipid that can be incorporated stably into liposomal membranes (Mamot et al. (2003) Cancer Res. 63: 3154-3161).

For encapsulation of doxorubicin, a remote-loading method utilizing triethylammonium sulfate was performed. Triethylammonium sulfate was prepared by simple titration of sulfuric acid with triethylamine. First, the dried lipids DSPC/cholesterol/PEG-DSPE (3:2:0.015, molar ratio) were dissolved in ethanol and heated to 60° C. The ethanolic lipid solution was subsequently injected into a heated solution (also 60° C.) of 200 mM triethylammonium sulfate (pH 5.5), followed by extrusion of the hydrated lipid suspensions at 60° C. through polycarbonate filters with ˜0.1 μm pore size. Free triethylammonium sulfate was removed by size-exclusion chromatography using a Sephadex G-75 column eluted with Hepes-buffered saline (5 mM Hepes (pH 6.5), 145 mM NaCl,). Liposomes were then incubated with doxorubicin for 30 min at 60° C., and unencapsulated doxorubicin was removed by gel-filtration chromatography using a Sephadex G-75 column. Liposome-encapsulated doxorubicin was then quantified by measuring absorbance at 498 nm following disruption of the liposomes using acidic isopropanol (90% (v/v) isopropanol, 10% (v/v) 0.1 M phosphoric acid).

Nanoliposomal topotecan (nLs-TPT) of an identical lipid composition was prepared using a novel intraliposomal drug stabilization strategy (Drummond et al. (2006) Cancer Res. 66: 3271-3277). Unlike the method utilized for encapsulation of doxorubicin above, the drug-entrapping solution employed for TPT was triethylammonium sucrose octasulfate (TEA₈SOS; 0.65 M TEA, pH 5.5). TEA8SOS was prepared from the commercially obtained sodium salt by ion-exchange chromatography on the Dowex 50Wx8-200 resin in the H⁺ form, immediately followed by titration with neat triethylamine. Following extrusion, unentrapped TEA₈SOS was removed on a Sepharose CL-4B size-exclusion column eluted with Hepes-buffered dextrose (5 mM Hepes, 5% (w/v) dextrose). Topotecan was then added at a TPT to PL ratio of 350 g TPT/mol PL and the pH adjusted to 6.0-6.5 with 1 M HCl before initiating loading at 60° C. for 30 min. The resulting nLs-TPT was quenched on ice for 15 min, followed by purification on a Sephadex G-75 column to remove unencapsulated TPT. A detailed description and characterization of these liposomes and the associated loading method for a different drug has been described elsewhere (Id.), and will be described in detail for topotecan elsewhere.

Immunoliposome Construction

To construct immunoliposomes, various scFvs were conjugated to β-(N-maleimido)propionyl poly(ethylene glycol)-1,2-distearoyl-3-sn-phosphoethanolamine (Mal-PEG-DSPE) as described (Nellis et al. (2005) Biotechnol. Prog. 21: 205-220; Nellis et al. (2005) Biotechnol. Prog. 21: 221-232). The (scFv)₂ dimers were reduced with 20 mM mercaptoethylamine by incubation at 37° C. for 15 min in PBS (pH 6.0) deoxygenated with bubbling argon. Reduced scFv were recovered by purification on a Sephadex G-25 gel-filtration column eluted with Hepes-buffered saline (5 mM Hepes (pH 7.0), 145 mM NaCl). Reduction efficiencies were evaluated by SDS-PAGE, allowing comparison of reduced and dimerized scFv; typically 90% reduced scFv was observed. For incorporation into preformed liposomes, micellar solutions of Mal-PEG-DSPE, were inserted into liposomes by incubation at 60° C. for 30 min at the ratio of 0.5 mol % of the liposomal phospholipids. The pH was raised to 7.0 by addition of a small quantity of concentrated Hepes buffer (0.5 M, pH 7.0) and the insertion of scFv was initiated by the addition of the desired scFv at a ratio of 5-60 μg of scFv/μmol PL. The conjugates were attached to the outer lipid monolayer of preformed liposomal therapeutics or fluorescent liposomes via hydrophobic DSPE domains. Unincorporated conjugates, unconjugated scFv or scFv dimers, and any released free small-molecule drugs were separated from the resulting ILs using a Sepharose CL-4B gel-filtration column eluted with Hepes-buffered saline, pH 6.5.

Internalization of Anti-EGFR Immunoliposomes

scFv-mediated internalization of fluorescent immunoliposomes was analyzed and quantified by microscopy and flow cytometry. C10 scFv fragments were inserted into liposomes at ratios of 5, 10, 15, 30 and 60 μg scFv/μmol phospholipid (Mamot et al. (2003) Cancer Res. 63: 3154-3161). scFv densities of 16, 32, 48, 96 and 192 scFv/liposome were calculated on the basis of the molecular mass of scFv (26 kDa) and the approximate number of phospholipid molecules/liposome (80,000). The conjugation technology for scFv to Mal-PEG-DSPE is remarkably reproducible with an efficiency of 77.3(±3.1) % (range 75-82% over six batches) (Nellis et al. (2005) Biotechnol. Prog. 21: 205-220). The final antibody densities on the resulting liposomes on the basis of this average efficiency are 12, 25, 37, 74, and 148 scFv/liposome. For microscopy studies, 150,000 cells were incubated with 50 μM PL of untargeted and EGFR-targeted ILs labeled with DiIC18(3)-DS in a 12-well plate for 2 h at 37° C., followed by washing with PBS and further incubation at 37° C. for 2 h. The cells were then analyzed by using an inverted fluorescence microscope (Nikon Eclipse, TE300) with a 540/25 nm band-pass filter for excitation and a long-pass filter at 565 nm for emission. For quantitative uptake of immunoliposomes, 250,000 cells were incubated with EGFR-targeted ILs labeled with ADS645 in a six-well plate for 2 h at 37° C., washed with PBS, removed in trypsin solution and fluorescence quantified with a FACSAria instrument (Beckton Dickinson). To determine the effect of EGF competition on internalization of EGFR-targeted ILs, IL internalization was quantified as described above, except that 50 μM targeted ILs and cells were incubated with EGF concentrations ranging from 0.2-25 nM. ANOVA (analysis of variance) was used to analyze the statistical differences of the quantified uptake.

Measurement of Immunoliposome Binding Affinity for Cells Expressing EGFR

Human breast cancer MDAMB468 cells expressing EGFR were grown to 80-90% confluence in L15 medium supplemented with 10% FBS and harvested by trypsinization. ILs labeled with DiIC18(3)-DS were incubated overnight with 10⁴ cells at concentrations of IL ranging from 0.31-2.5 nM. The concentration of IL was converted from the concentration of phospholipid on the basis of the approximate number of phospholipid molecules per liposome (80,000). Cell binding was performed at 4° C. in FACS buffer in adequate volume to ensure a fivefold excess of ILs and that the reaction had come to equilibrium. After two washes with 200 μl of FACS buffer, the amount of cell-bound ILs was quantified by flow cytometry in a FACS LSRII (Becton Dickinson). Data analysis was the same as for the measurement of scFv K_{D}.

Cytotoxicity of Chemotherapeutic-Containing Immunoliposomes

Specific cytotoxicity of EGFR-targeted ILs containing topotecan (IL-TPT) was evaluated in target cells in 96-well plates at a density of 10,000 cells/well for MDAMB468 breast carcinoma cells, and 3000 cells/well for U87vIII glioblastoma cells. After growth overnight, ILs or control treatments were applied to cells for 2 h at 37° C., followed by washing with PBS and addition of growth medium. Cells were additionally incubated at 37° C. for three days and analyzed for cell viability using 3-(4,5-dimethylthiazol-2-yl)-2,5-diphenyltetrazolium bromide staining (Carmicahael et al. (1987) Cancer Res. 47: 936-942).

### Results

Generation of a Library of Anti-EGFR C10 scFv Mutants

For affinity maturation, we used the internalizing EGFR scFv antibody C10 generated by selection of a non-immune human phage antibody library on EGFR over-expressing tumor cells (Heitner et al. (2001) J. Immunol. Methods, 248: 17-30). This scFv bound recombinant EGFR by ELISA, bound EGFR-expressing A431 cells with a K_{D} of 217 nM, and was rapidly endocytosed into EGFR-expressing cells (Id.). To generate a library of C10 scFv mutants, the C10 scFv gene was cloned into the yeast display vector pYD2 for display with a C-terminal SV5 epitope tag (Razai et al. (2005) J. Mol. Biol. 351: 158-169). The C10 scFv displayed at high levels on the yeast surface, with greater than 70% of yeast having detectable surface display, as detected by binding of SV5 antibody to the C-terminal epitope tag. However, when stained with biotinylated recombinant EGFR, there was only minimal binding at concentrations as high as 8 μM, and it was not possible to calculate a K_{D} (data not shown). This unexpected low level of binding was due to the low functional concentration of EGFR ECD, especially after biotinylation (EGFR ECD was estimated to be 2-10% active for mAb binding; see Materials and Methods). To randomly diversify the C10 scFv gene at a moderate mutation rate (Daugherty et al. (2000) Proc. Natl. Acad. Sci. USA, 97: 2029-2034), the scFv gene was amplified for 20 cycles under error-prone conditions with Taq polymerase and MnCl₂, followed by further amplification using a proof-reading polymerase for 35 cycles. The mutated scFv gene repertoire was cloned into the pYD2 vector using homologous recombination in Saccharomyces cerevisiae to generate a library of 5×10⁵ transformants. DNA sequencing of five scFv genes showed that each scFv gene had on average 3.8 amino acid substitutions (range 0-9). The location of the mutations was random, as the mutations were distributed evenly between V_{H} and V_{L} genes, and appeared in both the complementarity-determining regions (CDRs) and framework (FR) regions.

Isolation of C10 Mutants with Higher Affinity for EGFR

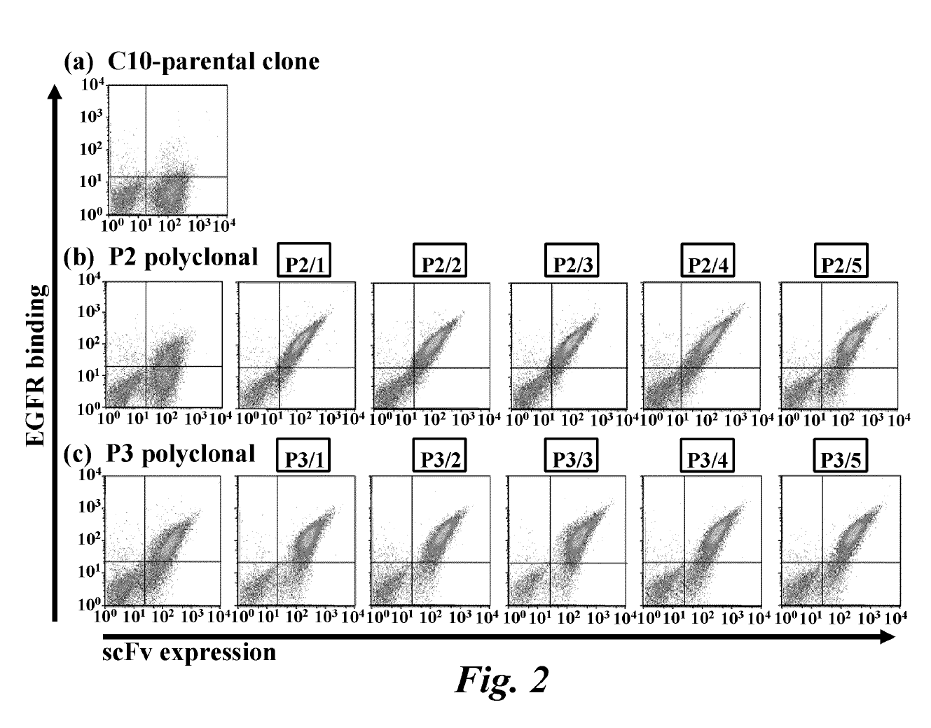

Higher-affinity C10 scFv mutants were selected from the error-prone yeast displayed library using flow cytometry (Feldhaus et al. (2003) Nature Biotechnol. 21: 163-170). Given the low-affinity binding of C10 scFv to recombinant EGFR ECD, 8 μM-biotinylated EGFR was used for the first round of sorting, with all yeast showing binding to EGFR above background selected for recovery (1.4% of the total population) (FIG. 1). To select for higher-affinity scFv, the concentration of EGFR ECD was decreased to 1 μM, 0.25 μM and 0.1 μM for the successive rounds of sorting. After four rounds of sorting, two gates were set to recover both the population with the highest mean fluorescent intensity (MFI) for antigen binding (gate P2) and the more highly expressed binding population (P3) (FIG. 1). The outputs from each of these sorting gates were grown, induced, stained with EGFR, and the MFI for EGFR binding compared to the MFI for EGFR binding of wild-type C10 scFv. The polyclonal populations from both sort gates showed significantly stronger EGFR staining than that of the C10 scFv (FIG. 2). Five monoclonal scFv from the P2 and P3 populations were also stained, and similarly showed stronger EGFR binding than wild-type C10 scFv, suggesting that each of these scFv had higher affinity for EGFR than the parental C10 scFv (FIG. 2).

DNA sequencing of the monoclonal scFv revealed that the individual scFv from the P2 population were more diverse than those from the P3 population. Four of the five scFv sequenced from the P2 population had unique sequences, one of these (P2/5) had the same sequence as the dominant clone from the P3 population (FIG. 3). For the four scFv unique to the P2 population, P2/1 has six amino acid changes located only in the V_{H} gene; P2/2 and P2/3 have the same sequence, with one mutation in V_{H} CDR1, V_{H} CDR2, V_{H} FR3, and V_{L} FR3; and P2/4 has one Gly to Ala substitution in V_{H} CDR3 and one Pro to Ala substitution in V_{L} FR1 (FIG. 3). On the basis of the deduced amino acid sequences, four unique scFv (P2/1, P2/2, P2/4 and P3/5) were chosen for further characterization and engineering. For the P2/1, P2/2, P2/4 and P3/5 scFv, the equilibrium dissociation constant (K_{D}) for each of the scFv for recombinant EGFR ECD was determined using flow cytometry (Feldhaus et al. (2003) Nature Biotechnol. 21: 163-170), and ranged from indeterminable (for the P3/5 scFv) to 1.8 μM (Table 2). Since the EGFR utilized was determined to be only 10% immunoreactive by SPR in a BIAcore, these measured affinities are likely significantly worse than the actual K_{D} and are presented only to provide quantification of the relative affinities of the scFv. Since K_{D} could not be measured for wild-type C10 scFv, a comparison could not be made with the mutant scFv to determine how many fold higher their affinities were for recombinant EGFR ECD.

Combining Mutations from Individual scFv to Create Higher-Affinity scFv

Since each mutant scFv had more than one amino acid substitution, it was difficult to predict which mutation(s) contributed to the improvement in affinity. To determine the effect of the Gly to Ala mutation in the V_{H} CDR3 of scFv P2/4, this substitution was introduced into three scFv with improved affinity for EGFR (P2/1, P2/2 and P3/5) using site-directed mutagenesis. The mutants with this V_{H} CDR3 mutation from P2/4 were named 2124, 2224 and 3524. The affinity of these three combined scFv for recombinant EGFR ECD was three- to sixfold higher than the K_{D} of the parental scFv (Table 2).

TABLE 2
K_{D} of scFvs for biotinylated EGFR ECD and A431 cells

| | | K_{D} for | K_{D} for |
|---|---|---|---|
| | | biotin-EGFR | A431 cells |
| | | ECD (μM)* | (nM) |
| Wild-type | C10 | N/A | 263.67 |
| Primary | P2/1 | 3.2 | 14.81 |
| mutants | P2/2 | 1.8 | 17.01 |
| | P2/4 | 1.8 | 15.39 |
| | P3/5 | N/A | 88.24 |
| Combined | 2124 | 0.56 | 9.90 |
| mutants | 2224 | 0.6 | 0.94 |
| | 3524 | 1.24 | 7.47 |

*K_{D} was measured on scFv displayed on yeast.

Equilibrium Binding Constants for scFv Binding to EGFR-Over-Expressing Tumor Cells

Since the KD of the original scFv C10 and the mutant scFv P3/5 could not be determined using biotinylated EGFR-ECD (Table 2), we expressed and purified native scFv and measured the KD on EGFR-expressing tumor cells. These measurements allowed comparison of the cell binding affinity and the affinity of binding to recombinant ECD, and provided a more relevant binding constant for the impact of scFv affinity on tumor cell targeting. To generate soluble scFv, the scFv genes were subcloned from pYD2 into the bacterial secretion vector pSYN1 (Schier et al (1995) Immunotechnology, 1:73-81), which directed the scFv to the bacterial periplasm using the pelB leader. The scFv was harvested from the bacterial periplasm and purified by immobilized metal-affinity chromatography as described (Id.). Yields of scFv ranged from 0.5-1.5 mg/l of Escherichia coli culture. To ensure that the KD of monovalent scFv was determined, gel-filtration chromatography was performed as described (Schier and Marks (1996) Hum. Antibodies Hybridomas: 7: 97-105) to separate native monovalent scFv from dimeric scFv and aggregated scFv. scFv was used for cellular K_{D} measurements immediately after gel-filtration. The KD of the parental scFv C10 as measured on A431 tumor cells was 264 nM (Table 2), which is close to the KD value previously measured for C10 scFv binding to tumor cells (217 nM) (Heitner et al. (2001) J. Immunol. Methods, 248: 17-30). By comparison, the affinities of the primary scFv mutants P2/1, P2/2, P2/4 and P3/5 were 14.8 nM, 17 nM, 15 nM and 88 nM, respectively, representing a 3-18-fold improvement in affinity compared to C10 scFv. Combining the VH CDR3 Gly to Ala substitution with the sequence of the P2/1, P2/2 and P3/5 scFv yielded further improvements in binding affinity, with the KD of the combined clones ranging from 9.9-0.9 nM (Table 2). These values represent a 1.5 to 17-fold increase in affinity as a result of combining mutations, and a 280-fold increase in affinity for the 2224 scFv compared to the parental C10 scFv.

Binding Specificity of C10 and Higher-Affinity scFv

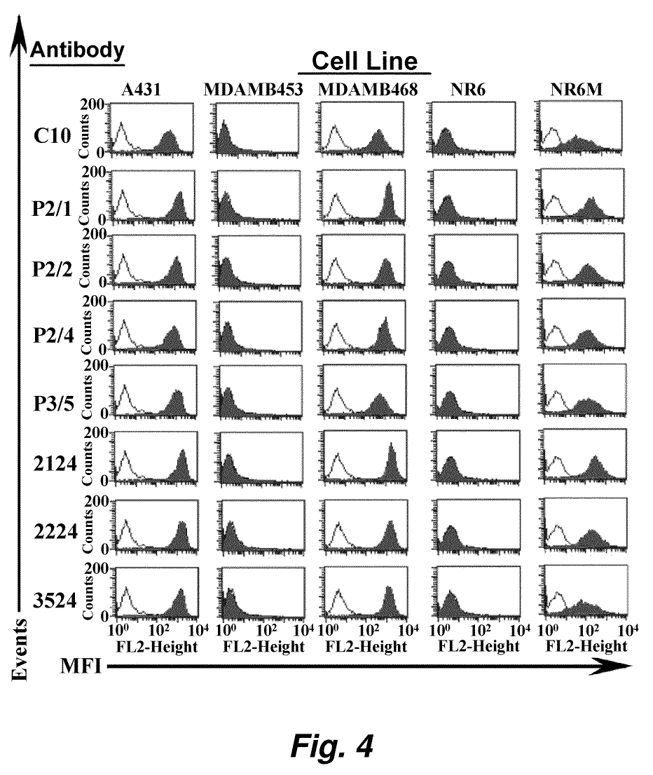

All the scFv mutants bound strongly to MDAMB468 and A431 cells, which express 1×10⁶-3×10⁶ EGFR/cell (FIG. 4) (Learn et al. (2004) Clin. Cancer Res. 10: 3216-3224; Milas et al. (2004) Int. J. Radiat. Oncol. Biol. Phys. 58: 966-971). Minimal binding above background was seen on MDAMB453, which express only 10⁴ EGFR/cell (FIG. 4). The binding of C10 scFv and C10 mutants to EGFR (vIII) stably transfected NR6M cells and parental NR6 cells was also measured. As expected, there was minimal binding of C10 scFv and C10 mutant scFv to NR6 cells, with strong staining of the NR6M cells with C10 scFv and the C10 scFv mutants (FIG. 4). The results confirm that C10 scFv binds both EGFR and the truncated form of EGFR, and that this specificity is maintained in the C10 mutant scFv.

Impact of scFv Affinity on Cell Binding and Uptake of EGFR-Targeted Immunoliposomes

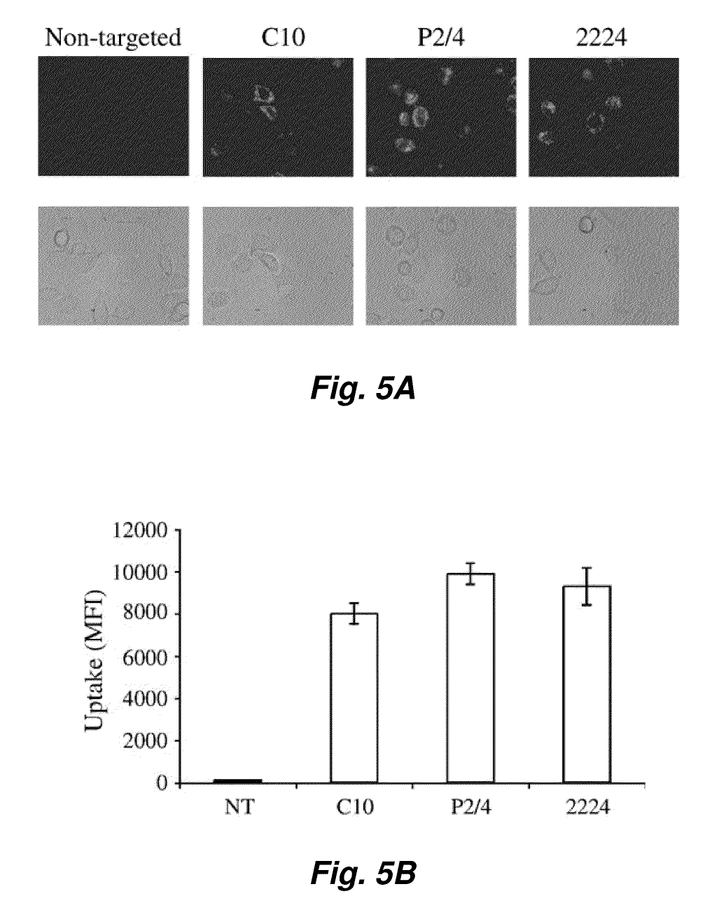

To determine the impact of intrinsic scFv affinity on the cellular uptake of immunoliposomes (ILs), we constructed ILs with either C10 scFv (KD=264 nM) or C10 mutant scFv P2/4 (KD=15.4 nM) or 2224 (KD=0.94 nM) on the liposome surface. EGFR immunoliposomes were generated by covalently attaching scFv to liposomes containing the fluorescent dye 1,1′dioctadecyl-3,3,3′,3′-tetramethylindocarbocyanine-5,5′-disulfonic acid (DiIC18(3)-DS) at a density of 74 scFv/liposome. The quantity of ILs taken up by EGFR-over-expressing MDAMB468 cells at an IL concentration of 0.63 nM was then determined by flow cytometry and confirmed by fluorescence microscopy. Fluorescence microscopy showed that ILs constructed from C10, P2/4 and 2224 scFv were endocytosed efficiently into MDAMB468 with no apparent difference in uptake for the different scFv (FIG. 5A). Quantitatively, IL uptake was significantly greater (24%) for the higher-affinity P2/4 compared to C10, but there was no statistically significant difference between ILs constructed from 2224 scFv and ILs constructed from either C10 or P2/4 scFv (FIG. 5B). These results suggest that intrinsic scFv affinity had, at best, a minimal impact on IL uptake at these particular EGFR and scFv densities.

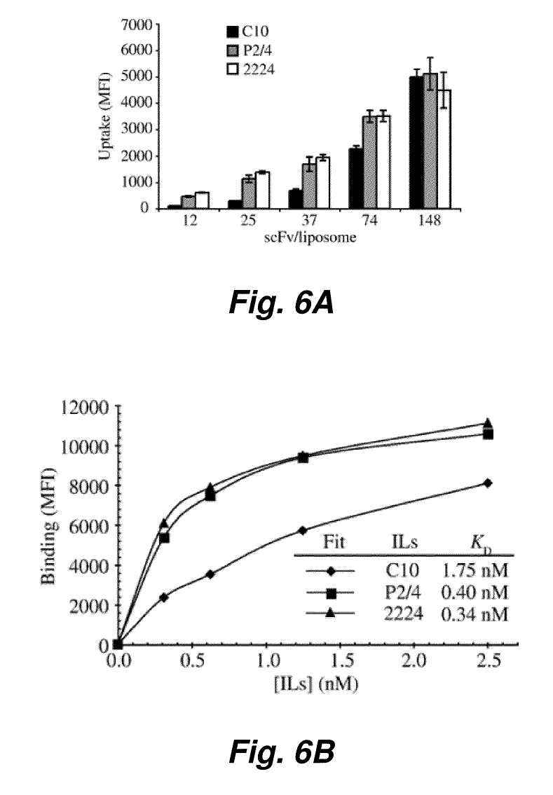

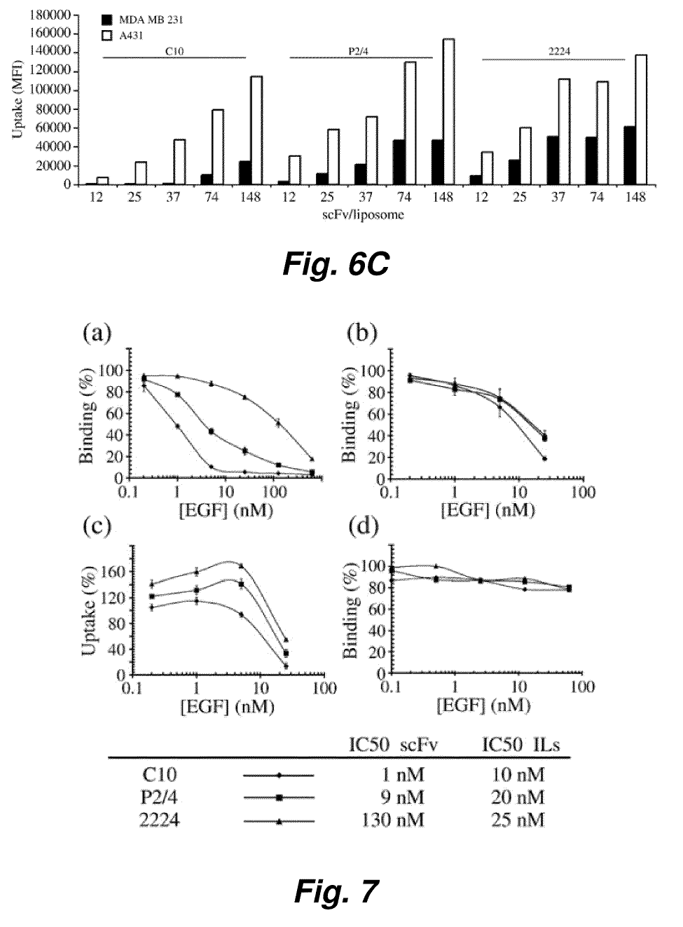

To study the interplay between IL concentration, liposomal scFv density, scFv affinity, and cellular uptake, we constructed a panel of ILs containing the fluorescent dye DiIC18(3)-DS at IL scFv densities ranging from 12 scFv/liposome to 148 scFv/liposome. At an scFv density of 148/liposome and an IL concentration of 0.63 nM, there was no significant difference in cellular IL uptake, consistent with the results presented above (FIG. 6A). In fact, there was a suggestion that uptake was less for ILs constructed using the highest affinity scFv. In contrast, at scFv densities of 74 scFv/liposome or less, there was less uptake of ILs constructed from the lowest affinity scFv (C10) compared to ILs constructed from the higher affinity scFv (FIG. 6A. To further elucidate the impact of IL concentration, scFv affinity and scFv density on IL binding and uptake, the apparent binding K_{D} of EGFR-targeted ILs on MDAMB468 cells was determined using flow cytometry (Table 3; FIG. 6B). At scFv densities of 12-37 scFv/liposome, the apparent K_{D} of C10 ILs could not be determined due to low binding signals and failure to reach surface saturation (data not shown). Where measurable, the apparent binding affinities of all three ILs, increased with higher scFv density, with the K_{D} of 2224 ILs ranging from 13.3 μM to 2 nM, with increasing liposomal scFv density. Thus IL scFv surface density had a much greater impact on cellular uptake than intrinsic scFv affinity.

TABLE 3
Apparent K_{D} of immunoliposomes constructed
with wild-type and affinity-matured scFv as measured on
MDAMB468 cells by flow cytometry

| | K_{D} (nM) | | | | |
|---|---|---|---|---|---|
| scFv/liposome | 12 | 25 | 37 | 74 | 148 |
| C10 | nd | nd | nd | 1.75 | 0.36 |
| P2/4 | nd | nd | 2.04 | 0.4 | 0.18 |
| 2224 | 13,364 | 11.76 | 2.08 | 0.34 | 0.22 |

nd, not determinable.

Impact of EGFR Density on Uptake of EGFR-Targeted Immunoliposomes

To determine the impact of receptor density on IL uptake, we measured the uptake of the different ILs into MDAMB231 cells which express 480,000 EGFR/cell as determined by flow cytometry using Quantum Simply Cellular anti-Human IgG beads (Bangs Laboratories Inc.). ILs constructed from C10, P2/4, or 2224 scFv had significantly less uptake into MDAMB231 cells compared to A431 cells at all scFv densities studied (FIG. 6C). Uptake into MDAMB231 cells increased with increasing scFv density, reaching a plateau at an IL scFv density of 74 scFv/IL for P2/4 scFv and at 37 scFv/IL for 2224 scFv (FIG. 6C).

Impact of Soluble EGF on Binding and Uptake of EGFR Immunoliposomes

Like cetuximab (C225), the natural ligand for EGFR (EGF) competes with C10 scFv for binding to EGFR. As a result, autocrine EGF produced at the tumor site could compete with C10 scFv mediated IL uptake. To determine the impact of EGF on scFv and IL binding as a function of scFv affinity, the effect of increasing concentrations of EGF on the binding and uptake of EGFR scFv and EGFR-targeted ILs was evaluated using flow cytometry. In MDAMB468 cells expressing intact EGFR, increasing concentrations of EGF reduced the binding of scFv, with the IC50 values (ligand value resulting in 50% inhibition of binding or uptake) differing by 130-fold, similar to the range of scFv affinities (FIG. 7(a)). In contrast, IL IC50 values differed by only 2.5-fold, ranging from 10 nM to 25 nM, despite the fact that intrinsic scFv affinity differed by 280 fold (FIG. 7(b)). Of note, the IC50 of the ILs constructed from the lowest affinity scFv (C10) was tenfold higher than the scFv IC50, while the ILs constructed from the highest affinity scFv (2224) was 20-fold less than the scFv IC50. The increase in IC50 for ILs constructed from the lowest affinity scFv probably results from slowing of the dissociation rate constant due to avidity from multiple scFv on the IL surface. The reason for the decrease in IC50 for ILs constructed from the highest affinity scFv is unclear, but probably results from a reduction in apparent affinity of the ILs compared to the scFv. The reduction is likely due to the association rate constant being slower, compared to the scFv due to their large size limiting diffusion. While IL binding to cells decreased with increasing EGF concentration, IL uptake into cells increased at low concentrations of EGF, suggesting a possible role of EGF in enhancing the turnover of EGF receptors and bound ILs on the cell surface (FIG. 7(c)). Truncation of the EGF binding epitope that occurs in EGFR (vIII) over-expressing U87vIII cells resulted in no effect of EGF on IL binding, as expected (FIG. 7(d)).

Impact of scFv Affinity on Cytotoxicity of Anti-EGFR Immunoliposomal Topotecan

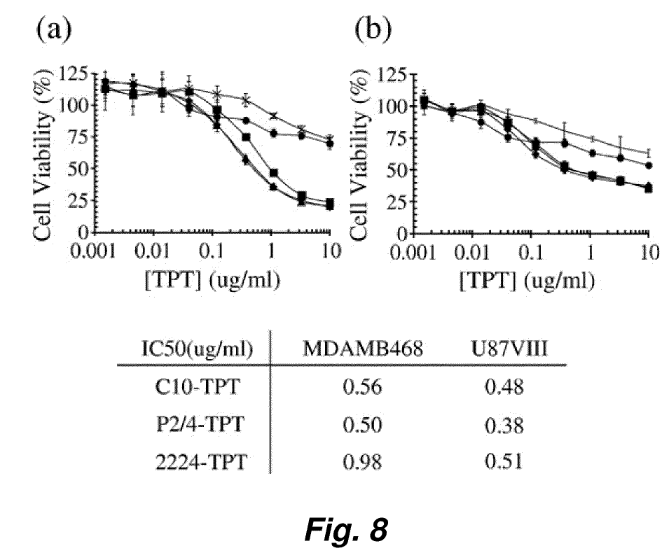

To determine the impact of scFv affinity on IL cytotoxicity, immunoliposomes containing the anticancer drug topotecan were constructed and evaluated in two EGFR-over-expressing cell lines: MDAMB468 breast carcinoma and U87vIII glioblastoma cells. Immunoliposomes were constructed using three different scFv mutants of varying affinity for EGFR: C10 (K_{D}=264 nM), P2/4 (K_{D}=15.4 nM), and 2224 (K_{D}=0.94 nM) and at a scFv/liposome density of 74. Similar to the results of uptake of fluorescent dye containing ILs, there was no difference in the cytotoxic effects of immunoliposomes constructed from the different affinity scFv on either EGFR overexpressing MDAMB468 cells (FIG. 8(a)) or EGFR-vIII-over-expressing U87vIII cells (FIG. 8(b)).

### Discussion

Antibodies and antibody fragments are being utilized increasingly in the treatment of cancer (Adams and Weiner (2005) Nature Biotechnol. 23:1147-1157). “Naked” antibodies, including trastuzumab (HER2) (Piccart-Gebhart et al (2005) N. Engl. J. Med. 353: 1659-1672), cetuximab (EGFR) (Cunningham et al (2004) N. Engl. J. Med. 351: 337-345), bevacizumab (VEGF) (Hurwitz et al. (2005) J. Clin. Oncol. 23: 3502-3508), alemtuzumab (CD52) (Wendtner et al. (2004) Leukemia, 18: 1093-1101), and rituximab (CD20) (Hainsworth et al. (2000) Blood, 95: 3052-3056) are already approved for use in oncology, and are an important component of clinical treatment strategies for various cancers. These antibodies act by using a variety of mechanisms to induce cytotoxic effects including activation of immune responses via complement-dependent or antibody-dependent cellular cytotoxicity, regulation of signal transduction pathways, inhibiting binding of receptor ligands, and modulating the activity of other therapeutic agents (Adams and Weiner (2005) Nature Biotechnol. 23: 1147-1157). Despite these therapeutic successes, response rates are still relatively modest, indicating that there is significant room for improvement in therapeutic efficacy. As a result, the next generation of “armed” antibodies have entered clinical trials, with strategies including conjugation of antibodies to small-molecule chemotherapeutic drugs, radioisotopes, enzymes, toxins, and nanocarriers such as liposomes, to specifically localize therapeutic agents at the site of the cancer (Noble et al. (2004) Expert Opin. Ther. Targets, 8: 335-353; Wu and Senter (2005) Nature Biotechnol. 23: 1137-1146). For example, we have recently employed immunoliposomes targeted against HER2/neu or EGFR to target a variety of drugs to tumors (Mamot et al. (2005) Cancer Res. 65: 11631-11638; Nielsen et al. (2002) Biochim. Biophys. Acta, 1591: 109-118; Drummond et al. (2005) Clin. Cancer Res. 11: 3392-3401; Park et al. (2002) Clin. Cancer Res. 8: 1172-1181), with improved antitumor efficacy upon molecular targeting being routinely observed.

Many of these strategies, including nanocarriers such as immunoliposomes, require antibodies that bind to receptors on tumor cells and are endocytosed, delivering the therapeutic agent into the cytosol. Phage antibody libraries have proven a useful resource for generating human scFv and Fab antibody fragments against therapeutic targets, including those on tumor cells (Sheets et al. (1998) [published erratum appears in Proc Natl Acad Sci USA 1999 January 1996(2):795]. Proc. Natl. Acad. Sci. USA, 95: 6157-6162; O'Connell (2002) J. Mol. Biol. 321:49-56; Schier et al. (1995) Immunotechnology, 1:73-81; Marks and Marks (1996) N. Engl. J. Med. 335: 730-733; Liu and Marks (2000) Anal. Biochem. 286: 119-128). Selection of antibodies by cell panning has been exploited to isolate cell-specific binders (Becerril et al. (1999) Biochem. Biophys. Res. Commun. 255: 386-393; Pereira et al. (1997) J. Immunol. Methods, 203: 11-24). For delivery of therapeutic agents into cells, it has proven possible to select specifically for phage antibodies capable of inducing receptor-mediated internalization (Becerril et al. (1999) Biochem. Biophys. Res. Commun. 255: 386-393). As a consequence, internalizing antibodies against ErbB2 and EGFR have been generated by recovering phage from within the cells (Poul et al. (2000) J. Mol. Biol. 301:1149-1161; Heitner et al. (2001) J. Immunol. Methods, 248: 17-30). The resulting antibody fragments are particularly suited for nanoparticle targeting, as the absence of the IgG crystallizable fragment (Fc) eliminates uptake by cellular Fc and complement receptors (Park et al. (2002) Clin. Cancer Res. 8: 1172-1181). Antibodies selected from non-immunized libraries, however, routinely possess binding affinities lower than those obtained using immunized libraries. For example, the internalizing HER2 scFv F5 has a K_{D} of 136 nM for cells over-expressing HER2, and the EGFR scFv C10 has a K_{D} of 217 nM for cells over-expressing EGFR (Heitner et al. (2001) J. Immunol. Methods, 248: 17-30; Neve et al. (2001) Biochem. Biophys. Res. Commun. 280: 274-279). While it is possible to increase antibody fragment affinity significantly using molecular evolution (Marks et al. (1992) Biotechnology (NY), 10: 779-783; Schier et al. (1996) J. Mol. Biol. 255: 28-43; Razai et al. (2005) J. Mol. Biol. 351: 158-169), this might not be necessary for nanoparticle targeting; antibody-targeted nanoparticles have multiple copies of antibody fragment on their surface, resulting in higher functional affinity due to avidity (Nielsen et al. (2000) Cancer Res. 60: 6434-6440; Adams et al. (2006) Clin. Cancer Res. 12:1599-1605). As a result, the impact of intrinsic antibody affinity on quantitative cellular uptake might be relatively unimportant. For example, the relatively low-affinity F5 can specifically target doxorubicin containing ILs to breast cancer cells in vitro and achieve therapeutic efficacy in vivo compared to untargeted liposomes (Nielsen et al. (2002) Biochim. Biophys. Acta, 1591: 109-118).

Here, we show that at a high liposomal surface density of scFv antibody fragment, there is no impact of intrinsic affinities between 264 nM and 0.9 nM on IL uptake into cells over-expressing EGFR. At lower surface scFv densities, there is less uptake of ILs targeted by the lowest affinity scFv (K_{D}=264 nM), but no difference in uptake between ILs targeted by 15 nM or 0.9 nM scFv. Thus, for tumor cells over-expressing EGFR, there appears to be a threshold intrinsic affinity of approximately 15 nM, above which there is no benefit of having a higher intrinsic affinity for IL uptake. Similar results are observed in tumor cells with lower levels of EGFR expression. Overall, IL uptake is lower than in cells expressing higher levels of EGFR, but with an intrinsic affinity greater than 15 nM, uptake reaches a plateau at an scFv density of 37-74 scFv/IL.

Similarly, functional avidity due to multicopy scFv display on the IL surface results in a minimal difference, 2.5-fold, in the inhibitory concentration of EGF required to block uptake of ILs constructed from scFv with affinities varying by 280-fold. In contrast, EGF competes uptake of the monomeric scFv at a level (130-fold difference) that is comparable to the differences in the intrinsic affinity (280-fold). Since there could be relatively high concentrations of EGF in the tumor microenvironment, the avidity effect should result in less inhibition of IL uptake compared to targeted drug carriers with fewer antibody copies. Finally, there was no difference in the in vitro cytotoxicity of ILs constructed from scFv with a K_{D} of 0.9 nM or 264 nM, consistent with the cellular uptake studies.

We did not study the relationship between intrinsic scFv affinity and in vivo efficacy of ILs. For monovalent scFv antibody fragments, tumor localization of anti-HER2 antibody fragment increases with increasing antibody fragment affinity, reaching a plateau at a K_{D} of 1 nM (Adams et al. (1998) Cancer Res. 58: 485-490). There is no increase in uptake at higher affinities (Adams et al. (2001) Cancer Res. 61: 4750-4755). For dimeric anti-HER2 diabody antibody fragments, there is no difference in tumor localization of diabodies constructed from scFv with intrinsic affinities ranging from 133 nM to 1 nM (Nielsen et al. (2000) Cancer Res. 60: 6434-6440). Like the multimeric ILs, the functional affinities of the bivalent diabodies were much more similar than the intrinsic affinities of the scFv from which they were constructed. On the basis of these studies, it might be expected that one would also observe no difference in therapeutic efficacy of the different EGFR-targeted ILs described here. In addition, the tumor localization of large macromolecular carriers, including liposomes, results more from the enhanced permeability and retention effect, whereby large liposomes become trapped in solid tumors due to the presence of a “leaky” microvasculature and the absence of functioning lymphatics (Drummond et al. (1999) Pharmacol. Rev. 51: 691-743; Matsumura and Maeda (1986) Cancer Res. 46: 6387-6392). Recent studies show that anti-HER2 immunoliposomes display a similar biodistribution, including tumor accumulation, to non-targeted liposomes and thus appear to be less dependent on molecular targeting for actual biodistribution and tumor localization in solid tumors (Kirpotin et al. (2006) Cancer Res. 66: 6732-6740). The therapeutic advantage of molecular targeting appears to arise from the intracellular uptake of the ILs compared to non-targeted liposomes. These studies would also support the argument that therapeutic efficacy of ILs will be relatively independent of the intrinsic antibody affinity.

In conclusion, we used yeast display and molecular evolution to construct a number of genetically related scFv mutants binding the same EGFR epitope in order to determine whether intrinsic antibody fragment affinity is an important determinant of nanoparticle uptake by tumor cells. Using ILs constructed from these mutants, we have shown that there is little impact of intrinsic affinity on the cellular binding, uptake, and in vitro cytotoxicity of EGFR-targeted ILs, especially once scFv affinity reaches 15 nM. There is no advantage in increasing affinity further in the system studied here, in which scFv surface density has a greater effect on cellular uptake.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1: A mutant C10 antibody that binds to human EGFR, said antibody comprising:
a heavy chain variable domain (VH) comprising the three VH CDRs of an antibody selected from the group consisting of P2/1, P2/2, P2/3, P2/4, 2124, 2224, and 3524.

2: A mutant C10 antibody that binds to human EGFR, said antibody comprising:
a light chain variable domain (VL) comprising the three VL CDRs of an antibody selected from the group consisting of P2/5, and P3/5.

3: The antibody of claim 1, wherein said antibody comprises the three VH CDRs and the three VL CDRs of an antibody selected from the group consisting of P2/1, P2/2, P2/3, P2/4, P2/5, 2124, 2224, 3524, P3/1, and P3/5.

4: The antibody of claim 3, wherein said antibody comprises the VH domain and the VL domain of an antibody selected from the group consisting of P2/1, P2/2, P2/3, P2/4, P2/5, 2124, 2224, 3524, P3/1, and P3/5.

5: The antibody according to claim 3, wherein said antibody is an antibody selected from the group consisting of an scFv, an IgG, a Fab, an (Fab′)₂, and an (scFv′)₂.

6. (canceled)

7: The antibody of claim 5, wherein said antibody is coupled to an effector.

8: The antibody of claim 7, wherein said effector comprises a moiety selected from the group consisting of a nanoparticle or microparticle, a microcapsule, a cytotoxin, a detectable label, a radionuclide, a drug, a liposome, a ligand, and an antibody.

9. (canceled)

10: The antibody of claim 7, wherein said effector comprises a liposome containing a moiety selected from the group consisting of an anti-cancer drug, a detectable label, and a radiosensitizing agent.

11: The antibody of claim 7, wherein said effector comprises a lipidic microparticle or nanoparticle.

12: The antibody of claim 11, wherein said microparticle or nanoparticle is selected from the group consisting of a liposome, a lipid-nucleic acid complex, a lipid-drug complex, a solid lipid particle, and a microemulsion droplet.

13-21. (canceled)

22: A method of inhibiting the growth or proliferation of a cancer cell expressing an epidermal growth factor receptor (EGFR), said method comprising contacting said cell with a composition comprising an antibody that binds an EGFR receptor, wherein said antibody comprises a heavy chain variable domain (VH) comprising the three VH CDRs of an antibody selected from the group consisting of P2/1, P2/2, P2/3, P2/4, 2124, 2224, and 3524; and/or a light chain variable domain (VL) comprising the three VL CDRs of an antibody selected from the group consisting of P2/5, and P3/5.

23: The method of claim 22, wherein said antibody comprises the three VH CDRs and the three VL CDRs of an antibody selected from the group consisting of P2/1, P2/2, P2/3, P2/4, P2/5, 2124, 2224, 3524, P3/1, and P3/5.

24: The method of claim 22, wherein said antibody comprises the VH domain and the VL domain of an antibody selected from the group consisting of P2/1, P2/2, P2/3, P2/4, P2/5, 2124, 2224, 3524, P3/1 and P3/5.

25: The method according to claim 23, wherein said antibody is an antibody selected from the group consisting of an scFv, an IgG, a Fab, an (Fab′)₂, and an (scFv′)₂.

26. (canceled)

27: The method of claim 25, wherein said antibody is coupled to an effector.

28: The composition of claim 7, wherein said effector comprises a moiety selected from the group consisting of a polymeric nanoparticle, a microcapsule, a cytotoxin, a radionuclide, a drug, a liposome, a ligand, and an antibody.

29: The composition of claim 7, wherein said effector comprises a moiety selected from the group consisting of a liposome, and a polymeric nanoparticle.

30-38. (canceled)

39: A method of detecting a cell expressing an epidermal growth factor receptor (EGFR) in a mammal, said method comprising:
contacting said cell with a composition comprising an antibody that binds an EGFR receptor, wherein said antibody comprises
a heavy chain variable domain (VH) comprising the three VH CDRs of an antibody selected from the group consisting of P2/1, P2/2, P2/3, P2/4, 2124, 2224, and 3524; and/or
a light chain variable domain (VL) comprising the three VL CDRs of an antibody selected from the group consisting of P2/5, and P3/5; and
wherein said antibody is attached to an epitope tag or a detectable label; and
detecting said epitope tag or said detectable label.

40: The method of claim 39, wherein said antibody comprises the three VH CDRs and the three VL CDRs of an antibody selected from the group consisting of P2/1, P2/2, P2/3, P2/4, P2/5, 2124, 2224, 3524, P3/1, and P3/5.

41: The method of claim 39, wherein said antibody comprises the VH domain and the VL domain of an antibody selected from the group consisting of P2/1, P2/2, P2/3, P2/4, P2/5, 2124, 2224, 3524, P3/1 and P3/5.

42: The method according to any one of claims 39-41, wherein said antibody is an antibody selected from the group consisting of an scFv, an IgG, a Fab, an (Fab′)₂, and an (scFv′)₂.

43-46. (canceled)

47: A method of delivering an effector into a cell expressing an epidermal growth factor receptor (EGFR), said method comprising:
contacting said cell with a composition comprising an effector attached to an antibody that binds an EGFR receptor, wherein said antibody comprises
a heavy chain variable domain (VH) comprising the three VH CDRs of an antibody selected from the group consisting of P2/1, P2/2, P2/3, P2/4, 2124, 2224, and 3524; and/or
a light chain variable domain (VL) comprising the three VL CDRs of an antibody selected from the group consisting of P2/5, P3/1, and P3/5; and
whereby said antibody is internalized into said cell.

48: The method of claim 47, wherein said antibody comprises the three VH CDRs and the three VL CDRs of an antibody selected from the group consisting of P2/1, P2/2, P2/3, P2/4, P2/5, 2124, 2224, 3524, P3/1 and P3/5.

49: The method of claim 47, wherein said antibody comprises the VH domain and the VL domain of an antibody selected from the group consisting of P2/1, P2/2, P2/3, P2/4, P2/5, 2124, 2224, 3524, P3/1 and P3/5.

50: The method according to claim 47, wherein said antibody is an antibody selected from the group consisting of an scFv, an IgG, a Fab, an (Fab′)₂, and an (scFv′)₂.

51. (canceled)

52: The composition of claim 47, wherein said effector comprises a moiety selected from the group consisting of a polymeric nanoparticle, a microcapsule, a cytotoxin, a radionuclide, a drug, a liposome, a ligand, and an antibody.

53-55. (canceled)
